(19) United States
(12) Patent Application Publication
(10) Pub. No.: US 2008/0152644 A1
(43) Pub. Date: Jun. 26, 2008

(54) Genes Involved in Neurodegenerative Conditions

(76) Inventors: Ho-Juhn Song, Andover, MA (US); Anju N. Kelkar, Sayville, NY (US); Dan Garza, Norfolk, MA (US); Mary Konsolaki, Westfield, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/817,358

(22) PCT Filed: Mar. 3, 2006
(86) PCT No.: PCT/US06/07645

§ 371 Date: Aug. 29, 2007

Related U.S. Application Data
(60) Provisional application No. 60/659,155, filed on Mar. 7, 2005.

(51) Int. Cl. A61K 39/00; G01N 33/53; C12Q 1/68

(52) U.S. Cl. 424/130.1; 435/7.1; 435/6

(57) ABSTRACT

The invention discloses suitable gene and polypeptide targets for the development of new therapeutics to treat, prevent or ameliorate neurodegenerative conditions. The invention also relates to methods to treat, prevent or ameliorate said conditions and pharmaceutical compositions therefor, as well as to a method to identify compounds with therapeutic usefulness to treat neurodegenerative conditions.

### FIELD OF THE INVENTION

This invention relates to genes involved in the development and/or progression of neurodegenerative conditions, specifically conditions involving the aberrant metabolism, trafficking or turnover of A-beta including, but not limited to, Alzheimer's Disease (AD). The invention also relates to the use of said genes as drug targets for the development of therapeutics useful to treat, prevent or ameliorate said neurodegenerative conditions.

### BACKGROUND OF THE INVENTION

AD is a progressive neurodegenerative disease that results in gradual cognitive and behavioral changes and loss of memory. See Selkoe, Physiol Rev, Vol. 81, No. 2, 741-766 (2001); Selkoe and Podlisny, Annu Rev Genomics Hum Genet, Vol. 3, No. 3, 67-99 (2002). Familial forms of AD have been linked to mutations in the gene that encodes amyloid precursor protein (APP). Differential cleavage of APP leads to production of 40 or 42 amino acid long peptides, designated as A-beta40 and A-beta42. A-beta40 is a non-amyloidogenic soluble form of Aβ and C99 is the β-secretase cleaved form of APP protein, which serves as the substrate for γ-secretase. APP mis-sense mutations are clustered around the A-beta cleavage sites and either increase the total production of A-beta-peptides or the A-beta42-/A-beta40-peptide ratio. Although both of these peptides are components of senile plaques (the neuropathological hallmark of AD), overproduction of A-beta42 is conducive to formation of amyloid plaques due to its hydrophobic nature and self-aggregation properties. Evin and Weidemann, Peptides, Vol. 23, No. 7, 1285-1297 (2002).

We have developed a model for A-beta-related toxicity in flies. See Finelli et al., Mol Cell Neurosci., Vol. 26, No. 3, 365 (2004); Iijima et al., Proc Natl Acad Sci USA. Vol. 101, No. 17, 6623 (2004), and this adult fly AD model that expresses A-beta42 in all neurons by using pan-neuronal GAL4 driver, ElavGal4C155 shows reduced short lifespan, progressive locomotion defect, olfactory associated learning and memory loss, progressive development of neuropathy (vacuolization in the adult brain) likely due to neuronal loss; these phenotypes that progressively increased with aging of adult flies was accompanied with A-beta aggregates and thyoflavin S-positive fibrilar tangles as can be found in human AD patient. Using this fly model we have conducted a genetic screen to look for modifiers of the A-beta42-dependent short lifespan phenotype. Our screen utilizes a publicly available collection of fly strains carrying independent insertions of the P-element in various regions of the fly genome. See Bier et al., Genes Dev., Vol. 3, 1273 (1989); Cooley et al., Science, Vol 239, 1121 (1988). The P-transposable element has been the vehicle most widely used to disrupt Drosophila genes because it inserts in heterochromatic as well as euchromatic regions, preferentially transposes near promoters and consequently it disrupts gene expression by its insertion. See Cooley et al., Science, Vol 239, 1121 (1988); reviewed in Bellen et al., Genetics, Vol. 167, 761 (2004). Therefore, we can achieve haplo-insufficiency of a gene expression after introduction of its mutant copy linked to P-element insertion. In order to carry out haplo-insufficiency genetic screen using P-elements, we crossed flies with one of P-elements to flies expressing A-beta42 by pan-neuronal Gal4 driver, ElavGal4C155 and obtained progeny from parental crosses and aged them until all progeny died in order to see whether gene liked to P-element is able to modify A-beta42 induced short lifespan. From this genetic screen we can determine genetic interactions that would affect the stability, aggregation, toxicity and/or secretion of the A-beta42-peptide, manifested as modification of the lifespan phenotype.

Applicants disclose herein surprising evidence suggesting that in our transgenic model, the A-beta42-peptide induces short lifespan along with AD pathology by the Drosophila neurons. Using this model system, Applicants have discovered and describe herein several new genes involved in the development and/or progression of AD. Thus, it is contemplated herein that these genes and the proteins encoded by these genes may serve as drug targets for the development of therapeutics to treat, prevent or ameliorate neurodegenerative conditions, specifically conditions involving, e.g., the aberrant metabolism, trafficking or turnover of A-beta including, but not limited to, AD.

### SUMMARY OF THE INVENTION

The instant application discloses human orthologs of several Drosophila genes as suitable targets for the development of new therapeutics to treat, prevent or ameliorate neurodegenerative conditions including, but not limited to, AD. Thus, in one aspect the invention relates to a method to identify modulators useful to treat, prevent or ameliorate said conditions comprising:

(a) assaying for the ability of a candidate modulator, in vitro or in vivo, to modulate a biological activity of a protein selected from the group consisting of the proteins disclosed in SEQ ID NOS:1-31 and/or modulate the expression of a gene encoding said protein; and which can further include

(b) assaying for the ability of an identified modulator to reverse the pathological effects observed in animal models of said neurodegenerative conditions and/or in clinical studies with subjects with said conditions.

In another aspect, the invention relates to a method to treat, prevent or ameliorate neurodegenerative conditions including, but not limited to, AD, comprising administering to a subject in need thereof an effective amount of a modulator of a protein selected from the group consisting of the proteins disclosed in SEQ ID NOS:1-31, wherein said modulator, e.g., inhibits or enhances a biological activity of said protein. In a further aspect, the modulator comprises antibodies to said protein or fragments thereof, wherein said antibodies can inhibit a biological activity of said protein in said subject.

In another aspect, the modulator inhibits or enhances the RNA expression of a gene encoding for a protein selected from the group consisting of the proteins disclosed in SEQ ID NOS:1-31. In a further aspect, the modulator comprises any one or more substances selected from the group consisting of antisense oligonucleotides, triple-helix DNA, ribozymes, RNA and DNA aptamers, siRNA and double- or single-stranded RNA, wherein said substances are designed to inhibit RNA expression of gene encoding said protein.

In another aspect, the invention relates to a method to treat, prevent or ameliorate neurodegenerative conditions including, but not limited to, AD, comprising administering to a subject in need thereof a pharmaceutical composition comprising an effective amount of a modulator of a protein selected from the group consisting of the proteins disclosed in SEQ ID NOS:1-31. In various aspects, said pharmaceutical composition comprises antibodies to said protein or fragments thereof, wherein said antibodies can inhibit a biological activity of said protein in said subject and/or any one or more substances selected from the group consisting of antisense oligonucleotides, triple-helix DNA, ribozymes, RNA and DNA aptamers, siRNA and double- or single-stranded RNA, wherein said substances are designed to inhibit RNA expression of gene encoding said protein. It is contemplated herein that one or more modulators of one or more of said proteins may be administered concurrently.

In another aspect, the invention relates to a pharmaceutical composition comprising a modulator to a protein selected from the group consisting of the proteins disclosed in SEQ ID NOS: 1-31 in an amount effective to treat, prevent or ameliorate a neurodegenerative condition including, but not limited to, AD, in a subject in need thereof. In one aspect, said modulator may, e.g., inhibit or enhance a biological activity of said protein. In a further aspect, said modulator comprises antibodies to said protein or fragments thereof, wherein said antibodies can, e.g., inhibit a biological activity of said protein.

In a further aspect, said pharmaceutical composition comprises a modulator which may, e.g., inhibit or enhance RNA expression of gene encoding said protein. In a further aspect, said modulator comprises any one or more substances selected from the group consisting of antisense oligonucleotides, triple-helix DNA, ribozymes, RNA or DNA aptamers, siRNA or double- or single-stranded RNA directed to a nucleic acid sequence of said protein, wherein said substances are designed to inhibit RNA expression of gene encoding said protein.

In another aspect, the invention relates to a method to diagnose subjects suffering from a neurodegenerative condition who may be suitable candidates for treatment with modulators to a protein selected from the group consisting of the proteins disclosed in SEQ ID NOS:1-31, comprising detecting levels of any one or more of said proteins in a biological sample from said subject wherein subjects with altered levels compared to controls would be suitable candidates for modulator treatment.

In another aspect, the invention relates to a method to diagnose subjects suffering from a neurodegenerative condition including, but not limited to, AD, who may be suitable candidates for treatment with modulators to a protein selected from the group consisting of the proteins disclosed in SEQ ID NOS:1-31, comprising assaying messenger RNA (mRNA) levels of any one or more of said protein in a biological sample from said subject, wherein subjects with altered levels compared to controls would be suitable candidates for modulator treatment.

In yet another aspect, there is provided a method to treat, prevent or ameliorate neurodegenerative conditions including, but not limited to, AD, comprising:

(a) assaying for mRNA and/or protein levels of a protein selected from the group consisting of the proteins disclosed in SEQ ID NOS:1-31 in a subject; and

(b) administering to a subject with altered levels of mRNA and/or protein levels compared to controls a modulator to said protein in an amount sufficient to treat, prevent or ameliorate said condition.

In particular aspects, said modulator inhibits or enhances a biological activity of said protein or RNA expression of gene encoding said protein.

In yet another aspect of the present invention, there are provided assay methods and diagnostic kits comprising:

(a) the components necessary to detect mRNA levels or protein levels of any one or more proteins selected from the group consisting of the proteins disclosed in SEQ ID NOS:1-31 in a biological sample, said kit comprising, e.g., polynucleotides encoding any one or more proteins selected from the group consisting of the proteins disclosed in SEQ ID NOS:1-31; and

(b) nucleotide sequences complementary to said protein;

(c) any one or more of said proteins, or fragments thereof of antibodies that bind to any one or more of said proteins, or to fragments thereof.

In a preferred aspect, such kits also comprise instructions detailing the procedures by which the kit components are to be used.

The present invention also pertains to the use of a modulator to a protein selected from the group consisting of the proteins disclosed in SEQ ID NOS:1-31, in the manufacture of a medicament for the treatment, prevention or amelioration of neurodegenerative conditions including, but not limited to, AD. In one aspect, said modulator comprises any one or more substances selected from the group consisting of antisense oligonucleotides, triple-helix DNA, ribozymes, RNA aptamer, siRNA and double- or single-stranded RNA, wherein said substances are designed to inhibit gene expression of said protein. In yet a further aspect, said modulator comprises one or more antibodies to said protein or fragments thereof, wherein said antibodies or fragments thereof can, e.g., inhibit a biological activity of said protein.

The invention also pertains to a modulator to a protein selected from the group consisting of the proteins disclosed in SEQ ID NOS:1-31 for use as a pharmaceutical. In one aspect, said modulator comprises any one or more substances selected from the group consisting of antisense oligonucleotides, triple-helix DNA, ribozymes, RNA aptamer, siRNA and double- or single-stranded RNA, wherein said substances are designed to inhibit gene expression of said protein. In yet a further aspect, said modulator comprises one or more antibodies to said protein or fragments thereof, wherein said antibodies or fragments thereof can, e.g., inhibit a biological activity of said protein.

Other objects, features, advantages and aspects of the present invention will become apparent to those of skill from the following description. It should be understood, however, that the following description and the specific examples, while indicating preferred aspects of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE FIGURES

FIG. 1. Typical parental crosses used for the P element based genetic screen to find modifiers of A-beta-induced lifespan phenotype. FM7, CyO, MKRS and TM6 are commonly used balancers for the X, 2^{nd} and 3^{rd} chromosomes, respectively.

FIG. 2. Lifespan of flies expressing A-beta42, A-beta40 or C99. Y axis represents the percentage of flies that were still alive at each time point. X axis represents the days at which flies were scored.

FIG. 3. Lifespan of flies expressing A-beta42, A-beta40 or C99 in glial cells.

FIG. 4. Lifespan of flies co-expressing A-beta42 and nep2.

FIG. 5. Lifespan of flies expressing A-beta42 that are haplo-insufficient for Tor function.

FIG. 6. Photomicrograph images of flies expressing A-beta42 that are haplo-insufficient for Tor function. The upper image is an original color photomicrograph of the results of the experiment. The lower image is a computer-generated grayscale image from the color original.

FIG. 7. Lifespan of flies expressing A-beta42 that have been fed various concentrations of the Tor inhibitor RAD001.

FIG. 8. Effect of Tor inhibitor RAD001 on the amount of total A-beta42 normalized to the total protein content of each brain extract.

### DETAILED DESCRIPTION OF THE INVENTION

All patent applications, patents and literature references cited herein are hereby incorporated by reference in their entirety.

Abbreviations used in the following description include:

| Abbreviation | Description |
|---|---|
| AD | Alzheimer's Disease |
| A-beta | A beta peptide |
| APP | Amyloid precursor protein |
| C99 | C-terminal 99 aa, [symbol] the secretase cleaved form of APP |
| | protein, which serves as the substrate for [symbol] secretase. |
| ELISA | Enzyme linked immunosorbent assay |
| EP | Expression P (element) |
| HIGS | haplo-insufficiency genetic screen |
| GFP | Green Fluorescent Protein |
| P-element | Drosophila transposable P-element |
| PBS | Phosphate buffered saline |
| RT | Room temperature |
| UAS | Upstream activating sequences |

In practicing the present invention, many conventional techniques in molecular biology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Edition, Cold Spring Harbor Laboratory Press, Cold Spring Harbor, N.Y. (1989); DNA Cloning: A Practical Approach, Vols. I and II, Glover, Ed. (1985); Oligonucleotide Synthesis, Gait, Ed. (1984); Nucleic Acid Hybridization, Hames and Higgins, Eds. (1985); Transcription and Translation, Hames and Higgins, Eds. (1984); Animal Cell Culture, Freshney, Ed. (1986); immobilized Cells and Enzymes, IRL Press (1986); Perbal, A Practical Guide to Molecular Cloning; the series, Meth Enzymol, Academic Press, Inc. (1984); Gene Transfer Vectors for Mammalian Cells, Miller and Calos, Eds., Cold Spring Harbor Laboratory Press, NY (1987); and Methods in Enzymology, Vols. 154 and 155, Wu and Grossman, and Wu, Eds., respectively (1987). Well-known Drosophila-molecular genetics techniques can be found, e.g., in Drosophila, A Practical Approach, Robert, Ed., IRL Press, Washington D.C. (1986).

Descriptions of flystocks can be found in the Flybase database at http://flybase.bio.indiana.edu.

Stock centers referred to herein include Bloomington and Szeged stock centers which are located at Bloomington, Ind., USA and Szeged, Hungary, respectively.

As used herein and in the appended claims, the singular forms “a”, “an” and “the” include plural reference unless the context clearly dictates otherwise. Thus, e.g., reference to “the antibody” is a reference to one or more antibodies and equivalents thereof known to those skilled in the art, and so forth.

“Nucleic acid sequence”, as used herein, refers to an oligonucleotide, nucleotide or polynucleotide, and fragments or portions thereof, and to DNA or RNA of genomic or synthetic origin that may be single- or double-stranded, and represent the sense or antisense strand.

The term “degenerate nucleotide sequence” refers to a sequence of nucleotides that includes one or more degenerate codons (as compared to a reference polynucleotide molecule that encodes a polypeptide). Degenerate codons contain different triplets of nucleotides, but encode the same amino acid residue, i.e., GAU and GAC triplets each encode Asp. Some polynucleotides encompassed by a degenerate sequence may have some variant amino acids, but one of ordinary skill in the art can easily identify such variant sequences by reference to the amino acid sequences encoding the proteins disclosed in SEQ ID NOS:1-31. Variants of the proteins disclosed in SEQ ID NOS:1-31 can be generated through DNA shuffling as disclosed by Stemmer, Nature, Vol. 370, No. 6488, 389-391 (1994); and Stemmer, Proc Natl Acad Sci USA, Vol. 91, No. 22, 10747-10751(1994). Variant sequences can be readily tested for functionality as described herein.

“Allelic variant” refers to any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

Allelic variants can be cloned by probing cDNA or genomic libraries from different individuals according to standard procedures. Allelic variants of the DNA sequences encoding proteins disclosed in SEQ ID NOS:1-31 and variants thereof, including those containing silent mutations and those in which mutations result in amino acid sequence changes, are within the scope of the present invention.

“Splice variant” refers to alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term “splice variant” is also used herein to denote a protein encoded by a splice variant of an mRNA transcribed from a gene.

The term “antisense”, as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. The designation “negative” is sometimes used in reference to the antisense strand, and “positive” is sometimes used in reference to the sense strand.

“cDNA” refers to DNA that is complementary to a portion of mRNA sequence and is generally synthesized from an mRNA preparation using reverse transcriptase.

As contemplated herein, antisense oligonucleotides, triple-helix DNA, RNA aptamers, ribozymes, siRNA and double- or single-stranded RNA are directed to a nucleic acid sequence such that the nucleotide sequence chosen will produce gene-specific inhibition of gene expression. For example, knowledge of a nucleotide sequence may be used to design an antisense molecule which gives strongest hybridization to the mRNA. Similarly, ribozymes can be synthesized to recognize specific nucleotide sequences of a gene and cleave it. See Cech, JAMA, Vol. 260, No. 20, 3030-3034(1988). Techniques for the design of such molecules for use in targeted inhibition of gene expression is well-known to one of skill in the art.

The individual proteins/polypeptides referred to herein include any and all forms of these proteins including, but not limited to, partial forms, isoforms, variants, precursor forms, the full-length protein, fusion proteins containing the sequence or fragments of any of the above, from human or any other species. Protein homologs or orthologs which would be apparent to one of skill in the art are included in this definition. These proteins/polypeptides may further comprise variants wherein the resulting polypeptide will be at least 80-90% or in other aspects, at least 95%, 96%, 97%, 98% or 99% identical to the corresponding region of a sequence selected from SEQ ID NOS:1-31. Percent sequence identity is determined by conventional methods. See, e.g., Altschul and Erickson, Bull Math Biol, Vol. 48, Nos. 5-6, 603-616 (1986); and Henikoff and Henikoff, Proc Natl Acad Sci U S A, Vol. 89, No. 22, 10915-10919 (1992). Briefly, two amino acid sequences are aligned to optimize the alignment scores using a gap opening penalty of 10, a gap extension penalty of 1, and the “BLOSUM62” scoring matrix of Henikoff and Henikoff. The percent identity is then calculated as:

(total number of identical matches)/(length of the longer sequence plus the number of gaps introduced into the longer sequence in order to align the two sequences)×100

It is also contemplated that the terms proteins or polypeptides refer to proteins isolated from naturally-occurring sources of any species, such as genomic DNA libraries, as well as genetically-engineered host cells comprising expression systems, or produced by chemical synthesis using, for instance, automated peptide synthesizers or a combination of such methods. Means for isolating and preparing such polypeptides are well-understood in the art.

The term “sample”, as used herein, is used in its broadest sense. A biological sample from a subject may comprise blood, urine, brain tissue, primary cell lines, immortalized cell lines or other biological material with which protein activity or gene expression may be assayed. A biological sample may include, e.g., blood, tumors or other specimens from which total RNA may be purified for gene expression profiling using, e.g., conventional glass chip microarray technologies, such as Affymetrix chips, RT-PCR or other conventional methods.

As used herein, the term “antibody” refers to intact molecules, as well as fragments thereof, such as Fa, F(ab′)₂ and Fv, which are capable of binding the epitopic determinant. Antibodies that bind specific polypeptides can be prepared using intact polypeptides or fragments containing small peptides of interest as the immunizing antigen. The polypeptides or peptides used to immunize an animal can be derived from the translation of RNA or synthesized chemically, and can be conjugated to a carrier protein. Commonly used carriers that are chemically coupled to peptides include bovine serum albumin and thyroglobulin. The coupled peptide is then used to immunize an animal, e.g., a mouse, goat, chicken, rat or a rabbit.

The term “humanized antibody”, as used herein, refers to antibody molecules in which amino acids have been replaced in the non-antigen binding regions in order to more closely resemble a human antibody, while still retaining the original binding ability.

A “therapeutically effective amount” is the amount of drug sufficient to treat, prevent or ameliorate a neurodegenerative condition, specifically a condition involving the aberrant metabolism, trafficking or turnover of A-beta including, but not limited to, AD.

A “transgenic” organism as used herein refers to an organism that has had extra genetic material inserted into its genome. As used herein, a “transgenic fly” includes embryonic, larval and adult forms of Drosophila that contain a DNA sequence from the same or another organism randomly inserted into their genome. Although Drosophila melanogaster is preferred, it is contemplated that any fly of the genus Drosophila may be used in the present invention.

As used herein, the term “A-beta” refers to beta- (β-) amyloid peptide which is a short (42 amino acid) peptide produced by proteolytic cleavage of APP by beta (β) and gamma (γ) secretases. It is the primary component of amyloid depositions, the hallmark of AD and the cause of neuronal cell death and degeneration. A-beta42 is provided herein as SEQ ID NO: 32. A-beta40 is constituted of residues 1-40 of the sequence shown in SEQ ID NO: 32. The sequence of C99 is given in SEQ ID NO:33.

As the term is used herein, the “reduced (short) lifespan” phenotype is characterized by expression of A-beta42 that leads 50% of adult Drosophila to die approximate around 21 days to 28 days compared to control flies that express A-beta40 and C99 or GFP and can be caused by degeneration of neuronal cells.

As used herein, “ectopic” expression of the transgene refers to expression of the transgene in a tissue or cell or at a specific developmental stage where it is not normally expressed.

As used herein, “phenotype” refers to the observable physical or biochemical characteristics of an organism as determined by both genetic makeup and environmental influences.

As used herein, “neurodegenerative conditions” include those conditions associated with progressive deterioration of the nervous system, caused, e.g., by errors in the regulation of the APP pathway, specifically, conditions involving, e.g., the aberrant metabolism, trafficking or turnover of A-beta including, but not limited to, AD.

“UAS region”, as used herein, refers to an UAS recognized by the Gal4 transcriptional activator.

As used herein, a “control fly” refers to fly that is of the same genotype as flies used in the methods of the present invention except that the control fly does not carry the mutation being tested for modification of phenotype.

As used herein, a “transformation vector” is a modified transposable element used with the transposable element technique to mediate integration of a piece of DNA in the genome of the organism and is familiar to one of skill in the art.

As used herein, “elevated transcription of mRNA” refers to a greater amount of mRNA transcribed from the natural endogenous gene encoding a protein, e.g., a human protein set forth in SEQ ID NOS:1-31, compared to control levels. Elevated mRNA levels of a protein, e.g., a human protein disclosed on SEQ ID NOS:1-31, may be present in a tissue or cell of an individual suffering from a neurodegenerative condition compared to levels in a subject not suffering from said condition. In particular, levels in a subject suffering from said condition may be at least about twice, preferably at least about five times, more preferably at least about 10 times, most preferably at least about 100 times the amount of mRNA found in corresponding tissues in humans who do not suffer from said condition. Such elevated level of mRNA may eventually lead to increased levels of protein translated from such mRNA in an individual suffering from said condition as compared to levels in a healthy individual.

As used herein, a “Drosophila transformation vector” is a DNA plasmid that contains transposable element sequences and can mediate integration of a piece of DNA in the genome of the organism. This technology is familiar to one of skill in the art.

Methods of obtaining transgenic organisms, including transgenic Drosophila, are well-known to one skilled in the art. For example, a commonly used reference for P-element mediated transformation is Spradling, Drosophila: A practical approach, Roberts, Ed., 175-197, IRL Press, Oxford, UK (1986). The EP element technology refers to a binary system, utilizing the yeast Gal4 transcriptional activator, that is used to ectopically regulate the transcription of endogenous Drosophila genes. This technology is described in Brand and Perrimon, Development, Vol. 118, No. 2, 401-415 (1993); and Rorth (1998), supra.

A “host cell”, as used herein, refers to a prokaryotic or eukaryotic cell that contains heterologous DNA that has been introduced into the cell by any means, e.g., electroporation, calcium phosphate precipitation, microinjection, transformation, viral infection and the like.

“Heterologous”, as used herein, means “of different natural origin” or represents a non-natural state. For example, if a host cell is transformed with a DNA or gene derived from another organism, particularly from another species, that gene is heterologous with respect to that host cell and also with respect to descendants of the host cell which carry that gene. Similarly, heterologous refers to a nucleotide sequence derived from and inserted into the same natural, original cell type, but which is present in a non-natural state, e.g., a different copy number, or under the control of different regulatory elements.

A “vector” molecule is a nucleic acid molecule into which heterologous nucleic acid may be inserted which can then be introduced into an appropriate host cell. Vectors preferably have one or more origin of replication, and one or more site into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes”.

“Plasmids” generally are designated herein by a lower case p preceded and/or followed by capital letters and/or numbers, in accordance with standard naming conventions that are familiar to those of skill in the art. Starting plasmids disclosed herein are either commercially-available, publicly-available on an unrestricted basis, or can be constructed from available plasmids by routine application of well-known, published procedures. Many plasmids and other cloning and expression vectors that can be used in accordance with the present invention are well-known and readily-available to those of skill in the art. Moreover, those of skill, readily may construct any number of other plasmids suitable for use in the invention. The properties, construction and use of such plasmids, as well as other vectors, in the present invention will be readily apparent to those of skill from the present disclosure.

The term “isolated” means that the material is removed from its original environment, e.g., the natural environment, if it is naturally-occurring. For example, a naturally-occurring polynucleotide or polypeptide present in a living animal is not isolated, but the same polynucleotide or polypeptide, separated from some or all of the co-existing materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

As used herein, the term “transcriptional control sequence” or “expression control sequence” refers to DNA sequences, such as initiator sequences, enhancer sequences and promoter sequences, which induce, repress or otherwise control the transcription of a protein encoding nucleic acid sequences to which they are operably-linked. They may be tissue specific and developmental-stage specific.

A “human transcriptional control sequence” is a transcriptional control sequence normally found associated with the human gene encoding a polypeptide set forth in SEQ ID NOS:1-31 of the present invention as it is found in the respective human chromosome.

A “non-human transcriptional control sequence” is any transcriptional control sequence not found in the human genome.

The term “polypeptide” is used, interchangeably herein, with the terms “polypeptides” and “protein(s)”.

A chemical derivative of a protein set forth in SEQ ID NOS:1-31 of the invention is a polypeptide that contains additional chemical moieties not normally a part of the molecule. Such moieties may improve the molecule's solubility, absorption, biological half-life, etc. The moieties may alternatively decrease the toxicity of the molecule, eliminate or attenuate any undesirable side effect of the molecule, etc. Moieties capable of mediating such effects are disclosed, e.g., in Remington's Pharmaceutical Sciences, 16^{th} Edition, Mack Publishing Co., Easton, Pa. (1980).

The ability of a substance to “modulate” a protein set forth in SEQ ID NOS:1-31 or a variant thereof, i.e., “a modulator of a protein selected from the group consisting of the proteins disclosed in SEQ ID NOS:1-31” includes, but is not limited to, the ability of a substance to inhibit or enhance the activity of said protein and/or variant thereof and/or inhibit or enhance the RNA expression of gene encoding said protein or variant. Such modulation could also involve affecting the ability of other proteins to interact with said protein, e.g., related regulatory proteins or proteins that are modified by said protein.

The term “agonist”, as used herein, refers to a molecule, i.e., modulator, which, directly or indirectly, may modulate a polypeptide, e.g., a polypeptide set forth in SEQ ID NOS:1-31 or a variant thereof, and which increases the biological activity of said polypeptide. Agonists may include proteins, nucleic acids, carbohydrates or other molecules. A modulator that enhances gene transcription or a biological activity of a protein is something that increases transcription or stimulates the biochemical properties or activity of said protein, respectively.

The terms “antagonist” or “inhibitor” as used herein, refer to a molecule, i.e., modulator, which directly or indirectly may modulate a polypeptide or variant thereof, e.g., a polypeptide set forth in SEQ ID NOS:1-31, which blocks or inhibits the biological activity of said polypeptide. Antagonists and inhibitors may include proteins, nucleic acids, carbohydrates or other molecules. A modulator that inhibits gene expression or a biological activity of a protein is something that reduces gene expression or biological activity of said protein, respectively.

As generally referred to herein, a “protein or gene selected from the group consisting of the proteins disclosed in SEQ ID NOS:1-31” refers to the human form of the protein or gene. It is recognized, that polypeptides (or nucleic acids which encode those polypeptides) containing less than the described levels of sequence identity to proteins in SEQ ID NOS:1-31 and arising as splice or allelic variants or that are modified by minor deletions, by conservative amino acid substitutions, by substitution of degenerate codons or the like, also are encompassed within the scope of the present invention. A variety of known algorithms are known in the art and have been disclosed publicly, and a variety of commercially-available software for conducting homology-based similarity searches are available and can be used to identify variants of proteins disclosed herein. Examples of such software includes, but are not limited to, FASTA (GCG Wisconsin Package), Bic_SW (Compugeh Bioccelerator), BLASTN2, BLASTP2, BLASTD2 (NCBI) and Motifs (GCG). The BLAST algorithm is described in Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402. Suitable software programs are described, e.g., in Guide to Human Genome Computing, 2^{nd} edition, Bishop, Ed., Academic Press, San Diego, Calif. (1998); and The Internet and the New Biology: Tools for Genomic and Molecular Research, American Society for Microbiology, Peruski, Jr. and Harwood Peruski, Eds., Washington, D.C. (1997).

“In vivo models of a neurodegenerative condition, specifically conditions involving the aberrant metabolism, trafficking or turnover of A-beta” include those in vivo models of neurodegenerative diseases, such as AD, familiar to those of skill in the art. Such in vivo models include, e.g., the mouse model of AD disclosed in WO 94/00569. In addition, numerous cell lines may be used as in vitro models of AD and are familiar to one of skill in the art including, e.g., the cell lines. See Xia et al., PNAS USA, Vol. 94, No. 15, 8208-8213 (1997).

The genes of the present invention were identified using a transgenic fly, Drosophila melanogaster, whose genome comprises a DNA sequence encoding A-beta. Conventional expression control systems may be used to achieve ectopic expression of proteins of interest, including the A-beta peptide. Such expression may result in the disturbance of biochemical pathways and the generation of altered phenotypes. One such expression control system involves direct fusion of the DNA sequence to expression control sequences of tissue-specifically-expressed genes, such as promoters or enhancers. A tissue-specific expression control system that may be used is the binary Gal4-transcriptional activation system. See Brand and Perrimon (1993), supra.

The Gal4 system uses the yeast transcriptional activator Gal4, to drive the expression of a gene of interest in a tissue-specific manner. The Gal4 gene has been randomly inserted into the fly genome, using a conventional transformation system, so that it has come under the control of genomic enhancers that drive expression in a temporal and tissue-specific manner. Individual strains of flies have been established, called “drivers”, that carry those insertions. See Brand and Perrimon (1993), supra.

In the Gal4 system, a gene of interest is cloned into a transformation vector, so that its transcription is under the control of the UAS and the Gal4-responsive element. When a fly strain that carries the UAS gene of interest sequence is crossed to a fly strain that expresses the Gal4 gene under the control of a tissue-specific enhancer, the gene will be expressed in a tissue-specific pattern.

In order to generate phenotypes that are easily visible in adult tissues and can thus be used in genetic screens, Gal4 “drivers” that drive expression in later stages of the fly development may be used in the present invention. Using these drivers, expression would result in possible defects in the wings, the eyes, the legs, different sensory organs and the brain. These “drivers” include, e.g., apterous-Gal4 (wings), elav-Gal4 (CNS), sevenless-Gal4, Gal4, eyGal4 and pGMR-Gal4 (eyes). Descriptions of the Gal4 lines and notes about their specific expression patterns is available in Flybase (http://flybase.bio.indiana.edu).

Various DNA constructs may be used to generate a transgenic Drosophila melanogaster. For example, the construct may contain the A-beta-sequence cloned into the pUAST vector (see Brand and Perrimon (1993), supra) which places the UAS up-stream of the transcribed region. Insertion of these constructs into the fly genome may occur through P-element recombination, Hobo element recombination [see Blackman et al., EMBO J, Vol. 8, No. 1, 211-217 (1989)], homologous recombination [see Rong and Golic, Science, Vol. 288, No. 5473, 2013-2018 (2000)] or other standard techniques known to one of skill in the art.

As discussed above, an ectopically-expressed gene may result in an altered phenotype by disruption of a particular biochemical pathway. Mutations in genes acting in the same biochemical pathway are expected to cause modification of the altered phenotype. Thus, the transgenic fly carrying the A-beta42-sequence is used to identify genes involved in the development and/or progression of neurodegenerative conditions, e.g., conditions involving the aberrant metabolism, trafficking or turnover of A-beta, such as AD, by crossing this transgenic fly with a fly containing a mutation in a known or predicted gene; and screening progeny of the crosses for flies that display quantitative or qualitative modification of the “lifespan” phenotype of the A-beta42 transgenic fly, as compared to controls.

This system is highly beneficial for the elucidation of the function of A-beta peptides, as well as the identification of endogenous genes whose encoded proteins that directly or indirectly interact with them. Mutations that can be screened include loss-of-function alleles of known genes, or deletion strains. In this way, genes involved in the development and/or progression of neurodegenerative conditions can be identified. These genes and the polypeptides encoded by these genes can then serve as targets for the development of therapeutics to treat such conditions.

Nucleic acid molecules of the human homologs of the target polypeptides disclosed herein may act as target gene antisense molecules, useful, e.g., in target gene regulation and/or as antisense primers in amplification reactions of target gene nucleic acid sequences. Further, such sequences may be used as part of ribozyme and/or triple-helix sequences or as targets for siRNA or double- or single-stranded RNA, which may be employed for gene regulation. Still further, such molecules may be used as components of diagnostic kits as disclosed herein.

In cases where an identified gene is the normal or wild type gene, this gene may be used to isolate mutant alleles of the gene. Such isolation is preferable in processes and disorders which are known or suspected to have a genetic basis. Mutant alleles may be isolated from individuals either known or suspected to have a genotype which contributes to disease symptoms related to neurodegenerative conditions including, but not limited to, AD. Mutant alleles and mutant allele products may then be utilized in the diagnostic assay systems described herein.

A cDNA of the mutant gene may be isolated, e.g., by using PCR, a technique which is well-known to those of skill in the art. In this case, the first cDNA strand may be synthesized by hybridizing an oligo-dT oligonucleotide to mRNA isolated from tissue known or suspected to be expressed in an individual putatively carrying the mutant allele, and by extending the new strand with reverse transcriptase. The second strand of the complementary (cDNA) is then synthesized using an oligonucleotide that hybridizes specifically to the 5′ end of the normal gene. Using these two primers, the product is then amplified via PCR, cloned into a suitable vector, and subjected to DNA sequence analysis through methods well-known to those of skill in the art. By comparing the DNA sequence of the mutant gene to that of the normal gene, the mutation(s) responsible for the loss or alteration of function of the mutant gene product can be ascertained.

Alternatively, a genomic or cDNA library can be constructed and screened using DNA or RNA, respectively, from a tissue known to or suspected of expressing the gene of interest in an individual suspected of or known to carry the mutant allele. The normal gene or any suitable fragment thereof may then be labeled and used as a probe to identify the corresponding mutant allele in the library. The clone containing this gene may then be purified through methods routinely practiced in the art, and subjected to sequence analysis as described above.

Additionally, an expression library can be constructed utilizing DNA isolated from or cDNA synthesized from a tissue known to or suspected of expressing the gene of interest in an individual suspected of or known to carry the mutant allele. In this manner, gene products made by the putatively mutant tissue may be expressed and screened using standard antibody screening techniques in conjunction with antibodies raised against the normal gene product, as described below. For screening techniques, see, e.g., Antibodies: A Laboratory Manual, Harlow and Lane, Eds., Cold Spring Harbor Press, Cold Spring Harbor, N.Y. (1988). In cases where the mutation results in an expressed gene product with altered function, e.g., as a result of a mis-sense mutation, a polyclonal set of antibodies are likely to cross-react with the mutant gene product. Library clones detected via their reaction with such labeled antibodies can be purified and subjected to sequence analysis as described above.

In another aspect, nucleic acids comprising a sequence encoding a polypeptide set forth in SEQ ID NOS:1-31 or a functional-derivative thereof, may be administered to promote normal biological activity, e.g., normal A-beta turnover, by way of gene therapy. Gene therapy refers to therapy performed by the administration of a nucleic acid to a subject. In this aspect of the invention, the nucleic acid produces its encoded protein that mediates a therapeutic effect by, e.g., promoting normal A-beta turnover.

Any of the methods for gene therapy available in the art can be used according to the present invention. Exemplary methods are described below.

In a preferred aspect, the therapeutic comprises a nucleic acid encoding any polypeptide given by SEQ ID NOS:1-31. Commonly the nucleic acid is part of an expression vector that expresses a protein given by SEQ ID NOS:1-31, a fragment or chimeric protein thereof and variants thereof in a suitable host. In particular, such a nucleic acid has a promoter operably-linked to a coding region encoding a protein of SEQ ID NOS: 1-31, said promoter being inducible or constitutive, and, optionally, tissue-specific. In another particular aspect, a nucleic acid molecule is used in which the protein coding sequences for any of SEQ ID NOS:1-31 and any other desired sequences are flanked by regions that promote homologous recombination at a desired site in the genome, thus providing for intrachromosomal expression of the nucleic acid encoding the particular protein. See Koller and Smithies, Proc Natl Acad Sci USA, Vol. 86, No. 22, 8932-8935 (1989); and Zijlstra et al., Nature, Vol. 342, No. 6248, 435-438 (1989).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific aspect, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment, e.g., a gene gun; Biolistic, Dupont, or coating with lipids or cell-surface receptors or transfecting agents, encapsulation in liposomes, microparticles or microcapsules, or by administering it in linkage to a peptide which is known to enter the nucleus, by administering it in linkage to a ligand subject to receptor-mediated endocytosis (see, e.g., U.S. Pat. Nos. 5,166,320; 5,728,399; 5,874,297 and 6,030,954, all of which are incorporated by reference herein in their entirety), which can be used to target cell types specifically expressing the receptors, etc. In another aspect, a nucleic acid-ligand complex can be formed in which the ligand comprises a fusogenic viral peptide to disrupt endosomes, allowing the nucleic acid to avoid lysosomal degradation. In yet another aspect, the nucleic acid can be targeted in vivo for cell specific uptake and expression, by targeting a specific receptor. See, e.g., PCT Publications WO 92/06180; WO 92/22635; WO 92/20316; WO 93/14188 and WO 93/20221. Alternatively, the nucleic acid can be introduced intracellularly and incorporated within host cell DNA for expression, by homologous recombination. See, e.g., U.S. Pat. Nos. 5,413,923; 5,416,260 and 5,574,205; and Zijlstra et al. (1989), supra.

In a specific aspect, a viral vector that contains a nucleic acid encoding a Polypeptide of SEQ ID NOS: 1-31 is used. For example, a retroviral vector can be used. See, e.g., U.S. Pate. Nos. 5,219,740; 5,604,090 and 5,834,182. These retroviral vectors have been modified to delete retroviral sequences that are not necessary for packaging of the viral genome and integration into host cell DNA. The nucleic acid for the Polypeptide of SEQ ID NOS:1-31 to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557 and 6,033,8843, all of which are incorporated by reference herein in their entirety.

Adeno-associated virus (AAV) has also been proposed for use in gene therapy. Methods for producing and utilizing AAV are described, e.g., in U.S. Pat. Nos. 5,173,414; 5,252,479; 5,552,311; 5,658,785; 5,763,416; 5,773,289; 5,843,742; 5,869,040; 5,942,496 and 5,948,675, all of which are incorporated by reference herein in their entirety.

Another approach to gene therapy involves transferring a gene to cells in tissue culture by such methods as electroporation, lipofection, calcium phosphate mediated transfection or viral infection. Usually, the method of transfer includes the transfer of a selectable marker to the cells. The cells are then placed under selection to isolate those cells that have taken up and are expressing the transferred gene. Those cells are then delivered to a patient.

The resulting recombinant cells can be delivered to a patient by various methods known in the art. In a preferred aspect, epithelial cells are injected, e.g., subcutaneously. In another aspect, recombinant skin cells may be applied as a skin graft onto the patient. Recombinant blood cells, e.g., hematopoietic stem or progenitor cells, are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type and include, but are not limited to, epithelial cells, endothelial cells, keratinocytes, fibroblasts, muscle cells, hepatocytes; blood cells, such as T lymphocytes, B lymphocytes, monocytes, macrophages, neutrophils, eosinophils, megakaryocytes, granulocytes; various stem or progenitor cells, in particular, hematopoietic stem or progenitor cells, e.g., as obtained from bone marrow, umbilical cord blood, peripheral blood, fetal liver, etc.

In a preferred aspect, the cell used for gene therapy is autologous to the patient.

In an aspect, in which recombinant cells are used in gene therapy, the nucleic acid of a polypeptide set forth in SEQ ID NOS:1-31 is introduced into the cells such that it is expressible by the cells or their progeny, and the recombinant cells are then administered in vivo for therapeutic effect. In a specific aspect, stem or progenitor cells are used. Any stem cells and/or progenitor cells that can be isolated and maintained in vitro can potentially be used in accordance with this aspect of the present invention. Such stem cells include, but are not limited to, hematopoietic stem cells (HSC), stem cells of epithelial tissues such as the skin and the lining of the gut, embryonic heart muscle cells, liver stem cells (see, e.g., WO 94/08598) and neural stem cells. See Stemple and Anderson, Cell, Vol. 71, No. 6, 973-985 (1992).

Epithelial stem cells (ESCs) or keratinocytes can be obtained from tissues, such as the skin and the lining of the gut by known procedures. See Rheinwald, Methods Cell Biol, Vol. 21A, 229-254 (1980). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture. See Pittelkow and Scott, Mayo Clin Proc, Vol. 61, No. 10, 771-777 (1986). If the ESCs are provided by a donor, a method for suppression of host versus graft reactivity, e.g., irradiation, drug or antibody administration to promote moderate immunosuppression, can also be used.

With respect to HSCs, any technique which provides for the isolation, propagation and maintenance in vitro of HSCs can be used in this aspect of the invention. Techniques by which this may be accomplished include:

(a) the isolation and establishment of HSC cultures from bone marrow cells isolated from the future host or a donor; or

(b) the use of previously established long-term HSC cultures, which may be allogeneic or xenogeneic.

Non-autologous HSC are used preferably in conjunction with a method of suppressing transplantation immune reactions of the future host/patient. In a particular aspect of the present invention, human bone marrow cells can be obtained from the posterior iliac crest by needle aspiration. See, e.g., Kodo, Gale and Saxon, J Clin Invest, Vol. 73, No. 5, 1377-1384 (1984). In a preferred aspect of the present invention, the HSCs can be made highly enriched or in substantially pure form. This enrichment can be accomplished before, during or after long-term culturing, and can be done by any techniques known in the art. Long-term cultures of bone marrow cells can be established and maintained by using, e.g., modified Dexter cell culture techniques [see Dexter et al., J Cell Physiol, Vol. 91, No. 3, 335-344 (1977)] or Witlock-Witte culture techniques. See Witlock and Witte, Proc Natl Acad Sci USA, Vol. 79, No. 11, 3608-3612 (1982).

In a specific aspect, the nucleic acid to be introduced for purposes of gene therapy comprises an inducible promoter operably-linked to the coding region, such that expression of the nucleic acid is controllable by controlling the presence or absence of the appropriate inducer of transcription.

A her aspect of the present invention relates to a method to treat, prevent or ameliorate neurodegenerative conditions including, but not limited to AD, comprising administering to a subject in need thereof an effective amount of a modulator of a protein selected from the group consisting of the proteins disclosed in SEQ ID NOS:1-31 and/or variants thereof. In one aspect, the modulator comprises one or more antibodies to said protein, variant or fragments thereof, wherein said antibodies or fragments thereof can inhibit a biological activity of said protein or variant in said subject.

Described herein are methods for the production of antibodies capable of specifically recognizing one or more differentially expressed gene epitopes. Such antibodies may include, but are not limited to, polyclonal antibodies, monoclonal antibodies (mAbs), humanized or chimeric antibodies, single-chain antibodies, Fab fragments, F(ab′)₂ fragments, fragments produced by a Fab expression library, anti-idiotypic (anti-Id) antibodies and epitope-binding fragments of any of the above. Such antibodies may be used, e.g., in the detection of a target protein in a biological sample, or alternatively, as a method for the inhibition of a biological activity of the protein. Thus, such antibodies may be utilized as part of disease treatment methods, and/or may be used as part of diagnostic techniques whereby patients may be tested, e.g., for abnormal levels of polypeptides set forth in SEQ ID NOS:1-31, or for the presence of abnormal forms of these polypeptides.

For the production of antibodies to the polypeptides given by SEQ ID NOS:1-31 or variants thereof, various host animals may be immunized by injection with these polypeptides, or a portion thereof. Such host animals may include but are not limited to rabbits, mice, goats, chickens and rats, to name but a few. Various adjuvants may be used to increase the immunological response, depending on the host species including, but not limited to, Freund's (complete and incomplete); mineral gels, such as aluminum hydroxide; surface active substances, such as lysolecithin, pluronic polyols, polyanions, peptides, oil emulsions, keyhole limpet hemocyanin and dinitrophenol; and potentially useful human adjuvants, such as bacille Calmette-Guerin (BCG) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with a polypeptide given by SEQ ID NOS:1-31, or a portion thereof, supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to, the hybridoma technique [see Kohler and Milstein, Nature, Vol. 256, No. 5517, 495-497 (1975) and U.S. Pat. No. 4,376,110]; the human B-cell hybridoma technique [see Kosbor et al., Immunol Today, Vol. 4, 72 (1983) and Cole et al., Proc Natl Acad Sci USA, Vol. 80, 2026-2030 (1983)]; and the EBV-hybridoma technique. See Cole et al., Monoclonal Antibodies And Cancer Therapy, Alan R. Liss, Inc., 77-969 (1985). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” [see Morrison et al., Proc Natl Acad Sci USA, Vol. 81, No. 21, 6851-6855 (1984); Neuberger, Williams and Fox, Nature, Vol. 312, No. 5995, 604-608 (1984); Takeda et al., Nature, Vol. 314, No. 6010, 452-454 (1985)] by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine nab and a human immunoglobulin constant region.

Alternatively, techniques described for the production of single-chain antibodies [U.S. Pat. No. 4,946,778; Bird, Science, Vol. 242, 423-426 (1988); Huston et al., Proc Natl Acad Sci USA, Vol. 85, No. 16, 5879-5883 (1988); and Ward et al., Nature, Vol. 334, 544-546 (1989)] can be adapted to produce differentially-expressed gene, single-chain antibodies. Single-chain antibodies are formed by linking the heavy- and light-chain fragments of the Fv region via an amino acid bridge, resulting in a single-chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932,448; 5,693,762; 5,693,761; 5,585,089; 5,530,101; 5,910,771; 5,569,825; 5,625,126; 5,633,425; 5,789,650; 5,545,580; 5,661,016 and 5,770,429, the disclosures of all of which are incorporated by reference herein in their entirety.

Antibody fragments that recognize specific epitopes may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed [see Huse et al., Science, Vol. 246, No. 4935, 1275-1281 (1989)] to allow rapid and easy identification of monoclonal Fab fragments with the desired specificity.

As contemplated herein, an antibody of the present invention can be preferably used in a diagnostic kit for detecting levels of a protein disclosed in SEQ ID NOS:1-31 or antigenic variants thereof in a biological sample, as well as in a method to diagnose subjects suffering from neurodegenerative conditions who may be suitable candidates for treatment with modulators to a protein selected from the group consisting of the proteins disclosed in SEQ ID NOS:1-31. Preferably, said detecting step comprises contacting said appropriate tissue cell, e.g., biological sample, with an antibody which specifically binds to a polypeptide given by SEQ ID NOS:1-31, or fragments or variants thereof and detecting specific binding of said antibody with a polypeptide in said appropriate tissue, cell or sample wherein detection of specific binding to a polypeptide indicates the presence of a polypeptide set forth in SEQ ID NOS:1-31 or a fragment thereof.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well-known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for a polypeptide given by SEQ ID NOS:1-31, or fragments or variants thereof.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, P-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of the Polypeptide of SEQ ID NOS:1-31 or variant which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually-detectable with a light microscope. Immunofluorescence and EIA techniques are both very well-established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

The pharmaceutical compositions of the present invention may also comprise substances that inhibit the expression of a protein disclosed in SEQ ID NOS:1-31 or variants thereof at the nucleic acid level. Such molecules include ribozymes, antisense oligonucleotides, triple-helix DNA, RNA aptamers, siRNA and/or double- or single-stranded RNA directed to an appropriate nucleotide sequence of nucleic acid encoding such a protein. These inhibitory molecules may be created using conventional techniques by one of skill in the art without undue burden or experimentation. For example, modifications, e.g., inhibition, of gene expression can be obtained by designing antisense molecules, DNA or RNA, to the control regions of the genes encoding the polypeptides discussed herein, i.e., to promoters, enhancers and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

Similarly, inhibition of gene expression may be achieved using “triple-helix” base-pairing methodology. Triple helix pairing is useful because it causes inhibition of the ability of the double-helix to open sufficiently for the binding of polymerases, transcription factors or regulatory molecules. Recent therapeutic advances using triplex-DNA have been described in the literature. See Gee et al., Molecular and Immunologic Approaches, Huber and Carr, Eds., Futura Publishing Co., Mt. Kisco, N.Y. (1994). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences. Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules. See Grassi and Marini, Ann Med, Vol. 28, No. 6, 499-510 (1996); and Gibson, Cancer Metastasis Rev, Vol. 15, No. 3, 287-299 (1996). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell. See Cotten and Birnstiel, EMBO J. Vol. 8, No. 12, 3861-3866 (1989). In particular, a ribozyme coding DNA sequence, designed according to conventional, well-known rules and synthesized, e.g., by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter, e.g., a glucocorticoid or a tetracycline response element, is also introduced into this construct so that ribozyme expression can be selectively controlled. For saturating use, a highly and constituently active promoter can be used. tDNA genes, i.e., genes encoding tRNAs, are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly, the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA [see Good et al., Gene Ther, Vol. 4, No. 1, 45-54 (1997)] that can specifically inhibit their translation.

Gene specific inhibition of gene expression may also be achieved using conventional double- or single-stranded RNA technologies. A description of such technology may be found in WO 99/32619, which is hereby incorporated by reference in its entirety. In addition, siRNA technology has also proven useful as a means to inhibit gene expression. See Cullen, Nat Immunol, Vol. 3, No. 7, 597-599 (2002); and Martinez et al., Cell, Vol. 110, No. 5, 563-574 (2002).

Antisense molecules, triple-helix DNA, RNA aptamers, dsRNA, ssRNA, siRNA and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters, such as T7 or SP6. Alternatively, cDNA constructs that synthesize antisense RNA constitutively or inducibly can be introduced into cell lines, cells or tissues.

Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well-known in the art.

Detection of mRNA levels of proteins disclosed herein may comprise contacting a biological sample or even contacting an isolated RNA or DNA molecule derived from a biological sample with an isolated nucleotide sequence of at least about 20 nucleotides in length that hybridizes under high-stringency conditions, e.g., 0.1×SSPE or SSC, 0.1% SDS, 65° C.) with the isolated nucleotide sequence encoding a polypeptide set forth in SEQ ID NOS:1-31. Hybridization conditions may be highly-stringent or less highly-stringent. In instances wherein the nucleic acid molecules are deoxyoligonucleotides (oligos), highly-stringent conditions may refer, e.g., to washing in 6×SSC/0.05% sodium pyrophosphate at 37° C. (for 14-base oligos), 48° C. (for 17-base oligos), 55° C. (for 20-base oligos) and 60° C. (for 23-base oligos). Suitable ranges of such stringency conditions for nucleic acids of varying compositions are described in Krause and Aaronson, Methods Enzymol, Vol. 200, 546-556 (1991) in addition to Maniatis et al., cited above.

In some cases, detection of a mutated form of the gene which is associated with a dysfunction will provide a diagnostic tool that can add to or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acids, in particular mRNA, for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled nucleotide sequences encoding a polypeptide disclosed in SEQ ID NOS:1-31 or variants thereof. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers, Larin and Maniatis, Science, Vol. 230, No. 4731, 1242-1246 (1985). Sequence changes at specific locations may also be revealed by nuclease protection assays, such as RNase and S1 protection or the chemical cleavage method. See Cotton et al., Proc Natl Acad Sci USA, Vol. 85, 4397-4401 (1985). In addition, an array of oligonucleotides probes comprising nucleotide sequence encoding the polypeptides given by SEQ ID NOS:1-31, or variants or fragments of such nucleotide sequences can be constructed to conduct efficient screening of, e.g., genetic mutations. Array technology methods are well-known and have general applicability and can be used to address a variety of questions in molecular genetics including gene expression, genetic linkage and genetic variability. See, e.g., Chee et al., Science, Vol. 274, No. 5287, 610-613 (1996).

The diagnostic assays offer a process for diagnosing or determining a susceptibility to disease through detection of mutation in the gene of a polypeptide set forth in SEQ ID NOS:1-31 by the methods described. In addition, such diseases may be diagnosed by methods comprising determining from a sample derived from a subject an abnormally decreased or increased level of polypeptide or mRNA. Decreased or increased expression can be measured at the RNA level using any of the methods well-known in the art for the quantitation of polynucleotides, such as, e.g., nucleic acid amplification, for instance, PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

The present invention also discloses a diagnostic kit for detecting mRNA levels (or protein levels) which comprises:

(a) a polynucleotide of a polypeptide set forth in SEQ ID NOS:1-31 or a fragment thereof;

(b) a nucleotide sequence complementary to that of paragraph (a);

(c) a polypeptide of SEQ ID NOS:1-31 of the present invention encoded by the polynucleotide of paragraph (a);

(d) an antibody to the polypeptide of paragraph (c); and

(e) an RNAi sequence complementary to that of paragraph (a).

It will be appreciated that in any such kit, any of the substances in (a), (b), (c), (d) or (e) may comprise a substantial component. Such a kit will be of use in diagnosing a disease or susceptibility to a disease, particularly to a neurodegenerative disease, such as AD.

The differences in the cDNA or genomic sequence between affected and unaffected individuals can also be determined. If a mutation is observed in some or all of the affected individuals but not in any normal individuals, then the mutation is likely to be the causative agent of the disease.

An additional aspect of the invention relates to the administration of a pharmaceutical composition, in conjunction with a pharmaceutically acceptable carrier, excipient or diluent, for any of the therapeutic effects discussed above. Such pharmaceutical compositions may comprise, for example, a polypeptide set forth in SEQ ID NOS:1-31, antibodies to that polypeptide, mimetics, agonists, antagonists, inhibitors or other modulators of function of a polypeptide given by SEQ ID NOS:1-31 or a gene therefore. The compositions may be administered alone or in combination with at least one other agent, such as stabilizing compound, which may be administered in any sterile, biocompatible pharmaceutical carrier including, but not limited to, saline, buffered saline, dextrose and water. The compositions may be administered to a patient alone, or in combination with other agents, drugs or hormones.

In addition, any of the therapeutic proteins, antagonists, antibodies, agonists, antisense sequences or other modulators described above may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment, prevention or amelioration of pathological conditions associated with abnormalities in the APP pathway. Using this approach, one may be able to achieve therapeutic efficacy with lower dosages of each agent, thus reducing the potential for adverse side effects. Antagonists, agonists and other modulators of the human polypeptides set forth in SEQ ID NOS:1-31 and genes encoding said polypeptides and variants thereof may be made using methods which are generally known in the art.

The pharmaceutical compositions encompassed by the invention may be administered by any number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well-known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato or other plants; cellulose, such as methyl cellulose, hydroxypropylmethyl-cellulose or sodium carboxymethylcellulose; gums including arabic and tragacanth; and proteins, such as gelatin and collagen. If desired, disintegrating or solubilizing agents may be added, such as the cross-linked polyvinyl pyrrolidone, agar, alginic acid or a salt thereof, such as sodium alginate.

Dragee cores may be used in conjunction with suitable coatings, such as concentrated sugar solutions, which may also contain-gum arabic, talc, polyvinylpyrrolidone, carbopol gel, polyethylene glycol, and/or titanium dioxide, lacquer solutions and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches; lubricants, such as talc or magnesium stearate; and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution or physiologically-buffered saline. Aqueous injection suspensions may contain substances that increase the viscosity of the suspension, such as sodium carboxymethyl cellulose, sorbitol or dextran. Additionally, suspensions of the active compounds may be prepared as appropriate oily injection suspensions. Suitable lipophilic solvents or vehicles include fatty oils, such as sesame oil; or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly-concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with many acids including, but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder that may contain any or all of the following: 1-50 mM histidine, 0.1-2% sucrose and 2-7% mannitol, at a pH range of 4.5-5.5, that is combined with buffer prior to use.

After pharmaceutical compositions have been prepared, they can be placed in an appropriate container and labeled for treatment of an indicated condition. Such labeling would include amount, frequency and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs or pigs. The animal model may also be used to determine the appropriate concentration range and route of administration. Such information can then be used to determine useful doses and routes for administration in humans.

A therapeutically-effective dose refers to that amount of active ingredient which ameliorates the symptoms or condition. Therapeutic efficacy and toxicity may be determined by standard pharmaceutical procedures in cell cultures or experimental animals, e.g., the dose therapeutically effective in 50% of the population (ED₅₀) and the dose lethal to 50% of the population (LD₅₀). The dose ratio between toxic and therapeutic effects is the therapeutic index, and it can be expressed as the ratio, LD₅₀/ED₅₀. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities and tolerance/response to therapy. Long-acting pharmaceutical compositions may be administered every 3-4 days, every week or once every two weeks depending on half-life and clearance rate of the particular formulation.

Normal dosage amounts may vary from 0.1-100,000 mg, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569 and 6,051,561.

### EXAMPLES

The following Examples illustrate certain aspects of the present invention. They do not in any way limit the scope of the invention as a whole.

Materials and Methods

DNA Constructs and Molecular Techniques

A Drosophila model of AD is described in detail in U.S. Patent Application Publication No. US20020174446; Finelli et al., Mol Cell Neurosci., Vol. 26, No. 3, 365 (2004); and Iijima et al., Proc Natl Acad Sci USA. Vol. 101, No. 17, 6623 (2004). Briefly, in an effort to mimic disease-specific A-beta42 over-expression, transgenic flies whose genome comprises the UAS-A-beta42 amyloid transgene are created using the GAL4 expression system in order to ectopically express the transgene in Drosophila postmitotic neuronal cells. In order to express the A-beta42-peptide in the Drosophila neurons, the A-beta42 sequence is cloned into the pUAS vector which is directed to where the GAL4 is expressed throughout the development of the central nervous system (CNS) including eye, as well as during adulthood, making it a suitable system for expression of A-beta42. Likewise, UAS-A-beta40 and UAS-C99 were made, respectively. Hereafter, transgenic flies for expression of A-beta42, A-beta40 and C99 are designated UAS-A-beta42^{H29.3}, UAS-A-beta40^{G68.2} and UAS-C99^{I8}, respectively. For neuronal expression of transgenes such as A-beta42, A-beta40 or C99, ElavGal4^{C155} was used, and for the control expressing GFP, UAS-GFP was used. Flies harboring both constructs were obtained from the Bloomington stock center, IN, USA.

EP transgenic flies can be obtained from the Szeged, Hungary stock center, and P flies from the Bloomington stock center, IN.

repoGal4 was Provided by Dr. Ulrike Gaul, The Rockefeller University, NY.

The UAS-A-beta42^{H29.3}, UAS-Abeta40^{G68.2} and UAS-C99^{I8} strains were generated in the laboratories of Novartis Institutes for Biomedical Research, Inc., using constructs prepared in the laboratory of Dr. P. Paganetti. In the fly model for AD (See Finelli et al., Mol Cell Neurosci., Vol. 26, No. 3, 365 (2004); Iijima et al., Proc Natl Acad Sci USA. Vol. 101, No. 17, 6623 (2004)), ectopic over-expression of A-beta42 disrupts normal fly lifespan and produces histological defects such as holes (vacuolization) in the brain, and the severity of the disruption depends on age of transgenic flies reflected by increased toxicity of A-beta-protein. For example, while young transgenic flies expressing A-beta42 have no defects in brain structure, it has been seen that old transgenic flies expressing A-beta42 have many holes in brain structure. Since such brain defect was not found in old flies expressing A-beta40 or C99, brain vacuolization is a phenotype specific to A-beta42, reflecting that unlike A-beta40 or C99, A-beta42 is toxic to neuronal cells. Approximately 50% of Flies expressing A-beta42 generally die around 21 to 28 days, which is designated as reduced lifespan compared to control flies such as those expressing A-beta40 or C99 whose 50% survival is up to 60 days. Flies expressing A-beta42 also displayed a progressive locomotion defect termed sluggishness, compared to those with A-beta40. Interestingly, flies expressing C99 displayed progressive spasm-like behavior over age.

Generation of Additional UAS-A-beta42 Strains

In order to generate A-beta42 transgenic strains with higher levels of peptide expression, remobilization of the A-beta42 transgene in the UAS-A-beta42^{H29.3} strain was done as described, for example, in Robertson, H. M., Preston, C. R., Phillis, R. W., Johnson-Schlitz, D., Benz, W. K., and Engels, W. R. (1988); A stable genomic source of P element transposase in Drosophila melanogaster; Genetics 118:461-470. 43 new strains were generated from the original UAS-A-beta42^{H29.3} strain. 17 independent lines were selected because they showed a rough eye phenotype when the transgene was expressed under the elavGal4 driver. Expression levels of the transgenes were analyzed by western analysis. Two of these strains, UAS-A-beta42^{HJ2.19} and UAS-A-beta42^{HJ2.23}, were used in this study.

Genetic Crosses, Analysis and Visualization of Phenotypes

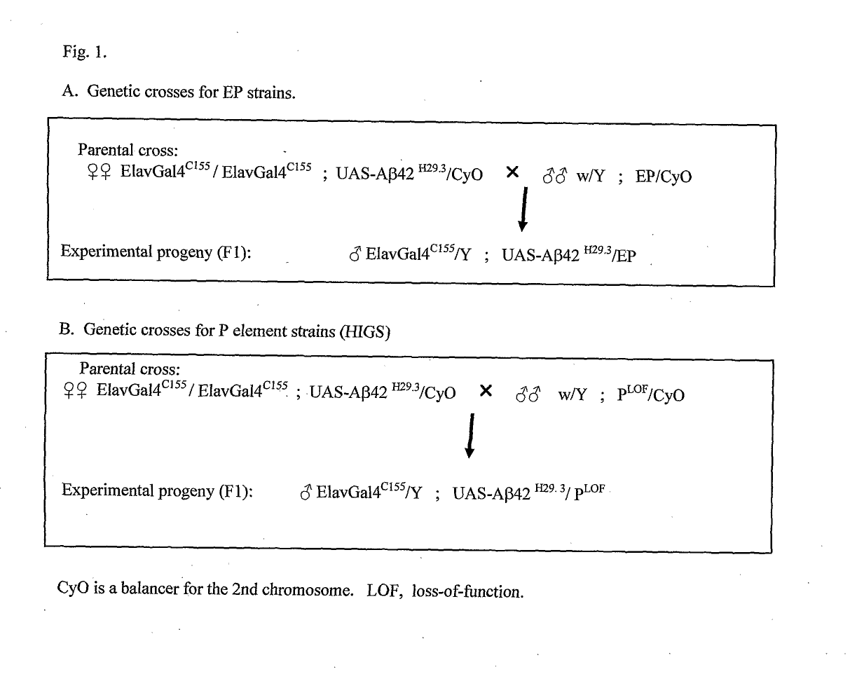

Flies were maintained in corn meal based standard fly food (Ashburner, 1989). Parental crosses were set up at 25° C. and F1 progeny was collected and raised at 29° C. (FIG. 1). Flies are crossed and maintained at 25° C. according to conventional methods except that all progeny are kept at 29° C. for maximal expression of phenotypes. In the binary Gal4 expression system, this temperature maximizes activity of the Gal4 protein. In the case of ElavGal4C¹⁵⁵/UAS-A-beta42, it is observed that the phenotype is stronger at 29° C., so these flies are kept at this temperature as well.

For 2^{nd} and 3^{rd} chromosome P-element strains, crosses were set using male flies (about 10) from the P-element strain and virgin females (about 10) from the A-beta42 over-expressing strain (ElavGal4C¹⁵⁵/UAS-A-beta42). For X-chromosome P strains, about 10 virgin females were collected from individual P strains and mated with 10 males from the A-beta over-expressing strain. Parental crosses were set up and raised at 25° C. 10 vials with 20 progeny from each cross were kept at 29° C. and scored for viability and behavior phenotypes as well as for general morphological changes. For scoring, live flies were transferred to fresh vials and the dead flies were counted every 2-3 days. For statistical evaluation of results, Log-Rank analysis, followed by chi-square comparison was performed, using the SAS 8.2 application.

For HIGS (Haplo-Insufficiency Genetics Screen), parental crosses of 10 males carrying P-element mutations (Spradling, A. C., Stern, D., Beaton, A., Rhem, E. J., Layerty, T., Mozden, N., Misra, S., and Rubin, G. M. (1999); The Berkeley Drosophila genome project gene disruption project; Single P-element insertions mutating 25% of vital Drosophila genes; Genetics 153: 135-177) and 10 female flies expressing A-beta42 were set up at 25° C., and 6 to 20 appropriate progeny were collected at day 14. The collected experimental progeny were kept at 29° C. and scored every 3-4 days for viability. If 50% of progeny of a cross that introduced a P-element mutation into A-beta42 expressing flies lived past 28 days they were considered to harbor a suppressor mutation, whereas if 50% of the progeny died by 14 days they were considered to harbor an enhancer mutation. For reexamination of modifier mutations and for background crosses, 100 progeny were scored.

For the locomotion assay, 20 flies of each experimental genotype were scored at 3, 10 and 15 days of age. Flies were transferred to fresh vials, tapped down and recorded for a few minutes until their climbing came to an equilibrium.

Western Blot Analysis

Flies of desired genotype were frozen in an Eppendorf tube using liquid nitrogen and quickly vortexed to sever the heads from the bodies. The contents of the tube were dumped on a weighing boat kept on dry ice and the heads were separated from other body parts using a pre-cooled fine paintbrush. To extract proteins from 50-100 heads, 50 μL of 28× stock of Complete Protease Inhibitor Mini tablets (Roche, Catalog No. 1 836 153) and 200 μL 2× sample buffer B (0.318 M Bicine, 30% sucrose, 2% SDS, 0.718 M Bistris) were added to the fly heads. Samples were subsequently homogenized by hand using a plastic pestle, then heated at 95° C. for 5 min. in a dry bath incubator and spun in a microcentrifuge at 12 K rpm, 5 min., 25° C. The supernatant was transferred to a protease free tube (Biopur, SRL, Rosario, Argentina) using a pipette tip. Protein samples were quantitated using the Biorad (Hercules, Calif.) protein assay (according to manufacturer's instructions for standard assay in a microtiter plate). Five percent (5%) 2-mercaptoethanol (2.5 μL for 50 μL) and 0.01% of Bromophenol blue (BB) (use 1 μL of 2% BB for 50 μL) were added to the samples. The samples were incubated at 100° C. in a dry bath incubator for 5 min. prior to loading. Fifty (50) μg of total protein extract is loaded for each sample, on a 15% tricine/tris SDS PAGE gel containing 8 M urea.

Samples were run at 40 V in the stacking gel and at 120 V in the separating gel (about 1.5 hours). One (1)×tris-tricine/SDS (diluted from 10× stock from Biorad) buffer is used as a cathode buffer between the gels and 0.2 M tris-HCl, pH 8.8 (diluted from 1.5 M stock from Biorad) is used as an anode buffer on the bottom. The A-beta-42 peptide control is human β-amyloid (1-42) (Biosource International, Camarillo, Calif., No. 03-111, Lot No. 0311219B). The peptide is dissolved at 1 μg/μL to make a stock. Prior to loading, an aliquot is diluted to 2 ng/μL concentration and mixed in 1:1 ratio with 2× sample buffer. Before loading, 2-mercaptoethanol and BB were added at 5% and 0.01%, respectively. Molecular weight marker RPN 755 (Amersham, Piscataway, N.J.) is used as a size marker. It is prepared for loading in a similar fashion to peptide marker. After electrophoresis, samples were transferred to PVDF membranes (Biorad, No. 162-0174) for 1 hour at 100 V and the membranes were subsequently boiled in 1×PBS for 3 min (with the membrane protein side down). The membranes were blocked with 5% non-fat milk prepwered in 1×PBS containing 0.1% Tween 20 for 1.5 hours to overnight. Antibody hybridization is as follows: the primary monoclonal antibody 6E10 (Senetek PLC, Napa, Calif.), which recognizes the first 19 amino acids of the A-beta-peptide, is used for probing (at a concentration of 1:1000) in 5% non-fat milk dissolved in 1×PBS containing 0.1% Tween-20, for 90 min. at RT. The membranes were washed 3× for 5 min., 15 min. and 15 min. each, in 1×PBS-0.1% Tween-20. The secondary antibody was anti-mouse antibody conjugated with horseradish peroxidase (Amersham Pharmacia Biotech, Piscataway, N.J., No. NA 931) and is used at 1:2000 in 5% non-fat milk dissolved in 1×PBS containing 0.1% Tween-20, for 90 min. at RT. Samples were washed as the after primary antibody incubation. ECL (Western Blotting Detection Reagents, Amersham Pharmacia Biotech, No. RPN2209) was used for detection. After blotting, membranes were washed with water several times and stained with Ponceau reagent to confirm equal loading in all lanes.

Immunostaining

Adult fly brains were dissected from aged adult fly of desired genotype in 1×PBS solution using a dissecting microscope and fixed in 4% paraformaldehyde (EMS, Washington, Pa.). Tissue was permeabilized in 1% Triton X-100 and 0.1 mg/ml RNase A in PBS overnight at room temperature and kept in the same solution under mild vacuum for 30, min to remove the air trapped within the tracheal system, followed by briefly washing in PBS, 3×5 min. Tissue was stained with 0.435 mM NBD-C₆-ceramide [6-((N-(7-nitrobenz-2-oxa-1,3-diazol-4-yl)amino)hexanoyl) sphingosin] and 0.1 mg/ml RNase A in 0.5% Tween 20 in water overnight at room temperature, followed by briefly washing in PBS, 3×5 min and then counterstained by 62.5 ug/ml propidium iodide (Molecular Probes, Eugene, Oreg.). Stained brains were cleared by incubation in FocusClear™ solution (PacGen, Vancouver, Canada), mounted in MountClear™ (PacGen, Vancouver, Canada) and analyzed using a Biorad confocal microscope and images are collected using Lasersharp 4.1 software (Biorad).

Sandwich ELISA

Antibodies: Mouse monoclonal antibody directed to the NH₂ terminus of the A-beta peptide was used as the capture antibody (Biosource Cat#44-352-100). The antibody was diluted in 1×PBS 1:2900 (3.5 μl/10 ml) and applied to Maxisorb Plates (Nunc Cat#442404) and 100 ul/96 well or 50 μl/384 well by incubating overnight at 4C. Polyclonal detection antibodies were obtained from Biosource (anti-hA-beta40 Cat#44-348 and anti-hA-beta42 Cat#44-344) and diluted 1/2000 in 1% BSA/PBS (1 μl/5 ml). The tertiary antibody (Santa Cruz Cat# SC2313) was a horseradish peroxidase labeled anti-rabbit IgG (Diluted 1/5000 in 1% BSA/PBS).

Preparation of the A-beta standards: 2.31 g of sodium bicarbonate was dissolved in 500 mL of distilled water and the pH was adjusted to 9.0 with 2N sodium hydroxide. The stock bicarbonate solution was filtered sterilized through a Millipore® 0.2 μm unit. Lyophilized A-beta standards were reconstituted in the bicarbonate solution to a concentration of 1 μg/mL (Biosource Cat#88-331, A-beta40; and Cat#88-332, A-beta42). The suspensions were mixed and transferred to ice for 90 minutes. The A-beta standards were diluted in 1% BSA/PBS containing 1 mM 4-(2-aminoethyl)-benzenesulfonylfluoride HCl (AEBSF) to 100,000 μg/mL, 10,000 pg/mL, 1000 pg/mL, 500 pg/mL, 250 pg/mL, 125 pg/mL, 62.5 pg/mL, 31.25 pg/mL, 15.63 pg/mL, and 0 pg/mL to generate a standard curve. For the preparation of the total A-beta peptide from fly heads expressing A-beta42, A-beta40 or GFP, 20 fly heads of 13 day old were obtained from each desired genotype subsequent to RAD001 treatment (10, 20, 30, 60 μM) at dry ice and homogenized by hand-pestle in ELISA buffer (see below) following boiling at 95° C. for 5 min.

Indirect Two Sandwich ELISA: After the overnight incubation with capture antibody the plates were washed twice on a microplate washer (Bioteck Instruments, Inc) in 1×PBS/0.05% Tween 20/1 mM EDTA). SuperBlock Buffer (Pierce Chemicals, Rockford, Ill., Cat #37515) was added 100 μl/96 well 50 μl/384 well and incubated 5 min RT shaking at 350 rpm. 100 μl of the transfected cell's conditioned media was removed and diluted 1:2 in 1×PBS/1% BSA containing 1 mM AEBSF and incubated at 4° C. overnight or at 20° C. for 2 hours on a shaker (300 rpm). The samples were removed and the plates were washed 4× with wash buffer. Detection antibody solution was added at 100 μl/well and the plates were incubated at room temperature for 2 hours while shaking. The plates were washed again 4× with wash buffer and the secondary antibody solution was added at 100 μl/well and incubated for 2 hours while shaking. The plates were washed 5× in wash buffer and pat dry on a paper towel. 100 μl of stabilized chromogen (tetramethylbenzidine, Biomedia Corp. #S18-100) was added to each well and the plate was incubated for 30 minutes in the dark. 100, of acid stop solution was added to the plates to stop the reaction. The plates were read on a microplate reader at 450 nM (Molecular Devices, Inc.) within one hour.

### Example 1

### Drosophila Model For the Primary Screen Using EP Insertion Lines

A Drosophila model for AD was created by over-expression of the A-beta42-peptide using ElavGal4C¹⁵⁵ (see methods section above and FIG. 1). This construct contained the A-beta42-coding region fused to the pre-proenkephalin signal peptide that has been shown to mediate secretion of A-beta42 from transfected mammalian cells. See Cescato (2000). Data previously indicate that A-beta42 effects in Drosophila are age-dependent. In order to be able to identify mutations that both enhance and suppress the A-beta42-phenotype, we chose to use for the genetic screen transgenic A-beta42 expression in all neurons. This A-beta42-strain, designated UAS-A-beta42^{H29.3}, carries A-beta42-transgenes on the 2^{nd} chromosome and ElavGal4C¹⁵⁵ on the X chromosome and shows a distinct adult lifespan phenotype at 25° C. This phenotype becomes more pronounced when adult flies are reared at 29° C. The temperature dependence of the lifespan phenotype makes the transgenic A-beta42 expressing adult fly suitable for our intended purposes.

### Example 2

### Phenotypes Caused by A-beta42 Over-Expression in the Adult Fly CNS

Based on the hypothesis that the expression of A-beta42 peptides in the CNS of adult flies could be toxic to neurons, we examined the lifespan and overall morphology of flies expressing A-beta42. The A-beta42 peptide was expressed using the binary Gal4/UAS expression system (Brand and Perrimon, 1993), with elavGal4^{C155}, which drives expression of Gal4 in all postmitotic neurons of the adult CNS. About 100 progeny were scored for each genotype. We found that flies expressing A-beta42 under the control of elavGal4C¹⁵⁵ did not show any changes in external morphology (including eye and bristle tissues), but did show reduced lifespan and progressive loss of locomotion activity.

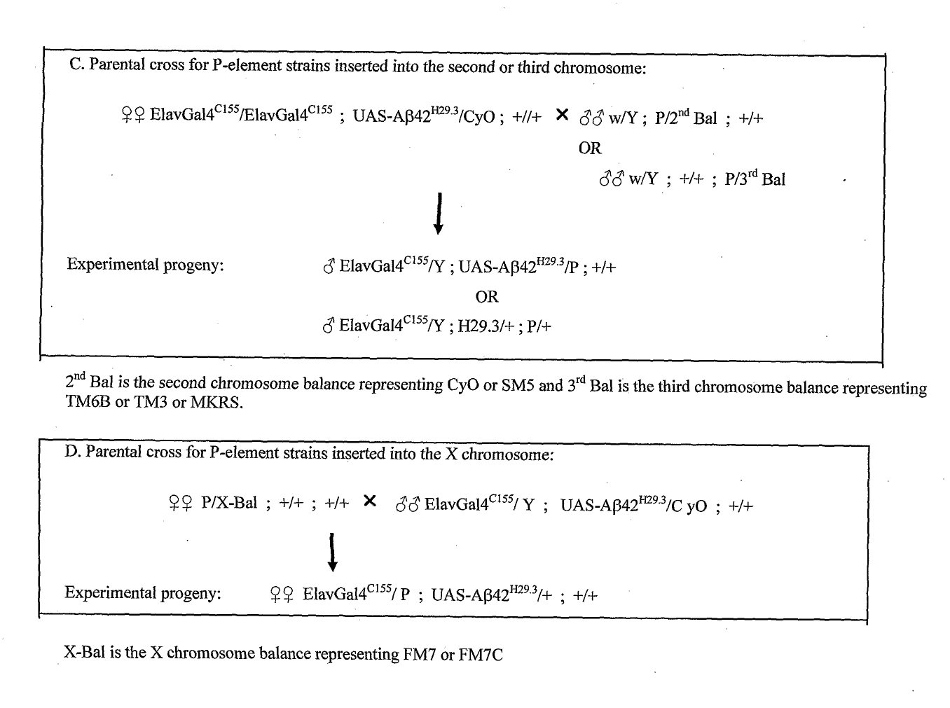

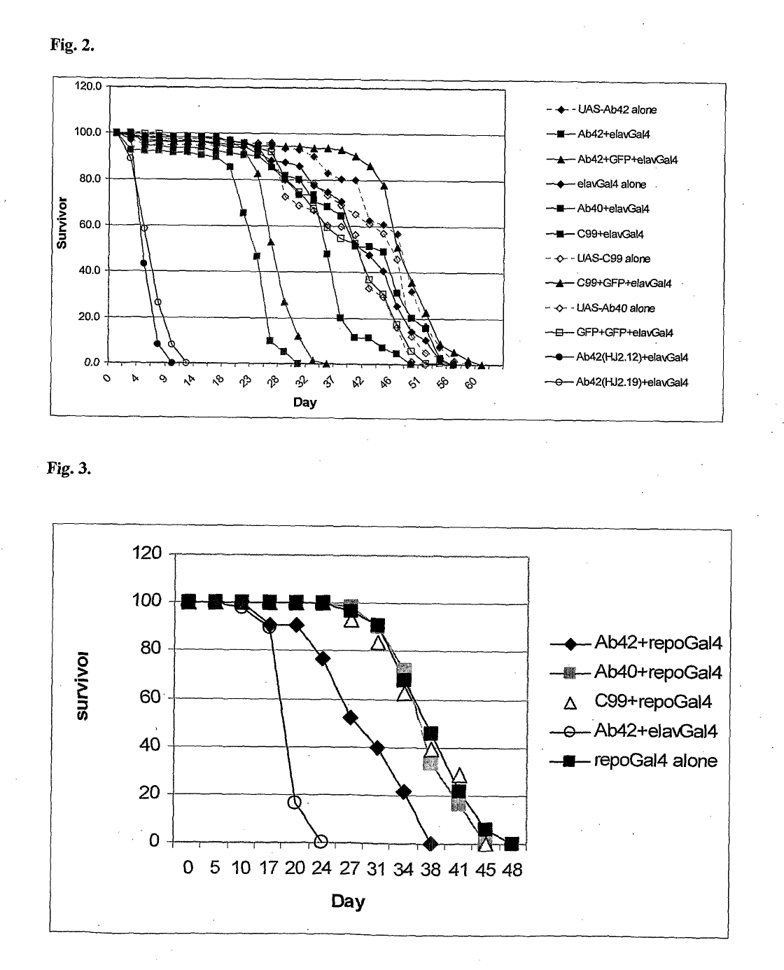

In particular, as seen in FIG. 2, flies expressing A-beta42^{H29.3} (A-beta42+elavGal4) or A-beta42^{H29.3} and GFP (A-beta42+GFP+elavGal4) died abruptly after day 19. Control flies expressing the elavGal4 driver alone (elavGal4 alone) or the UAS-A-beta42^{H29.3} transgene alone (UAS-A-beta42 alone) died around day 50. Higher expression of A-beta42 using A-beta42^{HJ2.12} and A-beta42^{HJ2.19} strains [A-beta42(HJ2.12), A-beta42(HJ2.19)] caused a much shorter lifespan. For statistical evaluation we used log-Rank analysis followed by chi-square comparison (P<0.001). As seen in FIG. 2, all other controls showed normal lifespan.

It is worth noting that adult flies co-expressing A-beta42 and GFP (control, FIG. 2) showed a small suppression of the lifespan phenotype, probably due to titration of the available Gal4 activity, caused by the presence of two copies of UAS constructs. No lethality was observed in the A-beta42 flies during development, suggesting that the observed phenotype is due primarily to A-beta42 toxicity manifested during adult stages.

We subsequently examined whether the shortened lifespan phenotype induced by A-beta42 depends on the dosage of A-beta42 peptide. We generated 43 new strains containing additional copies of A-beta42 by mobilizing the A-beta42 transgene in the original H29.3 strain (see Materials and Methods). In order to identify high level A-beta42 expressing lines within these new strains, we examined them for a rough eye phenotype. Since the original A-beta42 strain did not have a rough eye phenotype and we have previously shown that the A-beta42-induced rough eye phenotype is dose-dependent, we expected that this analysis would identify strains with higher expression levels of A-beta42. Indeed, two strains, A-beta42^{HJ2.12} and A-beta42^{HJ2.19}, were identified that showed rough eye phenotypes induced by the elavGal4 driver. Both of these strains also displayed much shorter lifespan (FIG. 2), suggesting that increased amounts of A-beta42peptides enhance its toxic effects.

In addition to the lifespan, we also found that A-beta42 expression caused progressive locomotion defects in adult flies. In order to analyze locomotion activity, we used a “climbing assay” (Le Bourg E, Lints F A. (1992). Hypergravity and aging in Drosophila melanogaster. 4. Climbing activity. Gerontology. 38:59-64), which measures the negative geotropic response that flies naturally display. Groups of flies at different stages of adult life (3, 10, 15 days) from experimental and control groups were assayed. We found that flies expressing A-beta42 showed reduced locomotion activity as they aged (Table 1). Three different UAS-A-beta42 strains were used, expressing different amounts of A-beta42 peptide (A-beta42^{H29.3}, A-beta42^{HJ2.12}, A-beta42^{HJ2.19}). Strains A-beta42^{HJ2.12} and A-beta42^{HJ2.19} express higher levels of the peptide than strain A-beta42^{H29.3}. As is seen in Table 1, the locomotion activity of A-beta42^{H29.3} flies was not significantly reduced until about 10-15 days. In contrast, flies expressing very high levels of A-beta peptide (A-beta42^{HJ2.12}, A-beta42^{HJ2.19}) had reduced locomotion already at day 3, suggesting that the locomotion defect is also dose dependent (Table 1).

TABLE 1
A-beta42 induced locomotion defect.

| Genotypes | 3 days | 10 days | 15 days |
|---|---|---|---|
| ElavGal4^{C155}; A-beta42^{H29.3} | ++++ | ++ | + |
| ElavGal4^{C155}; A-beta42^{HJ2.12} | ++ | nd | nd |
| ElavGal4^{C155}; A-beta42^{HJ2.19} | ++ | nd | |
| ElavGal4^{C155} | nd | nd | ++++ |
| ElavGal4^{C155}; A-beta42^{H29.3}; | ++++ | ++++ | +++ |
| EP3549 nep2 | | | |

++++, normal activity.
+++, slightly reduced activity.
++, very reduced activity.
nd, not determined.

Behavioral defects such as those observed in the lifespan and locomotion phenotypes have been previously associated with brain degeneration in flies (Min, K.-T., Benzer, S. 1999. Preventing neurodegeneration in the Drosophila mutant bubblegum. Science 284, 1985-1988; Wittmann, C. W., Wszolek, M. F., Shulman, J. M., Salvaterra, P. M., Lewis, J., Hutton, M., Feany, M. B. 2001. Tauopathy in Drosophila: neurodegeneration without neurofibrillary tangles. Science 293, 711-714; Jackson, J. R., Wiedau-Pazos, M., Sang, T. K., Wagle, N., Brown, C. A., Massachi, S. Geschwind, D. H. 2002. Human wild-type tau interacts with wingless pathway components and produces neurofibrillary pathology in Drosophila. Neuron 34, 509-519). Based on this, our studies show that A-beta induces neurodegenerative phenotypes in flies that are dose and age dependent.

### Example 3

### A-beta40 Over-Expression does not Lead to Any Lifespan Defects but C99 Expression Leads to a Novel Un-Coordinated Phenotype

In addition to A-beta42, we examined the effects of A-beta40 and C99 expression in flies. We found that flies expressing A-beta40 showed normal lifespan (FIG. 2), comparable to control flies expressing only the Gal4 driver. Since it has previously been shown by our collaborators at Novartis that these A-beta40 flies express comparable amounts of the peptide as A-beta42 flies (Y. Zhong, CSHL), these results suggest that A-beta40 has no toxic effects in the fly CNS.

In contrast, flies expressing C99 in the adult CNS displayed a novel un-coordinated locomotion phenotype (Table 2), whereas their lifespan was unaffected (FIG. 2). The un-coordinated phenotype caused the flies to be disoriented, and display a spasmodic movement, so that they were unable to respond normally in the climbing assay. The uncoordinated behavior lasted for a short time, after which the flies recovered fully. This phenotype was also age-specific, occurring first at 10 days of age, and becoming more severe flies aged (Table 2).

TABLE 2
C-99-induced locomotion defect.

| Genotypes | 3 days | 10 days | 15 days |
|---|---|---|---|
| ElavGal4^{C155}; C99¹⁸ | ++++ | +++ | ++ |
| ElavGal4^{C155} | nd | nd | ++++ |

++++, normal activity
+++, slightly uncoordinated activity
++, strong uncoordinated activity
nd, not determined

We have shown previously that expression of C99 in the neuromuscular junction causes the appearance of extra synaptic boutons (data not shown). Similar phenotypes have been described for mutations in genes that affect potassium channels (shaker, ether a go-go) and in general participate in pathways involved in postsynaptic potential (rutabaga, an adenylate cyclase; dunce, a cAMP-specific phosphodiesterase; Zhong, Y., Budnik, V., and Wu, C. F. (1992). Synaptic plasticity in Drosophila memory and hyperexcitable mutants: role of cAMP cascade. J. Neurosci. 12:644-51). Based on these studies, we postulate that the C99-induced uncoordinated phenotype might be the consequence of an effect on synaptic potential.

### Example 4

### Overexpression of A-beta42 in Adult CNS Glial Cells Also Causes Lifespan and Behavioral Phenotypes in Flies

In order to examine the effects of A-beta42 expression in a non-neuronal cell type of the brain, we expressed our transgenes specifically in glial cells. In order to achieve this, we used a Gal4 driver under the control of the repo (reversed polarity) regulatory sequences. Repo is exclusively expressed in most glial cells, the major non-neuronal type in the adult CNS (Xiong et al., 1994).

Flies expressing A-beta42 in glial cells under the control of repoGal4 showed reduced lifespan, compared to control flies expressing the repoGal4 driver alone (P<0.01), or those expressing A-beta40 or C99 (FIG. 3); however, the reduced lifespan phenotype was less severe than what was observed when A-beta42 was expressed in neuronal cells (A-beta42+elavGal4). It should be recalled that it is difficult to directly compare effects using the two drivers elavGal4 and repoGal4, since they have different cell type specificity and expression levels. Our experiments nevertheless suggest that A-beta42 is likely to be toxic in non-neuronal cells as well.

### Example 5

### Overexpression of a Fly Homologue of Human Neprilysin 2 Partially Rescues the Lifespan and Behavior Phenotypes

Neprilysin belongs to a family of Zn metallopeptidases that have been shown to degrade A-beta peptides (reviewed in Carson and Turner 2002). In this study, we examined the ability of a mutation that causes upregulation of a Drosophila neprilysin homolog (nep2) to modify the altered locomotion and reduced viability phenotypes caused by expression of A-beta42 in the fly CNS. The EP3549 mutation was used to upregulate nep2 expression. Expression P (EP) has similar genes as P-element except Gal4 binding sites (UAS). Nep2 gene expression was upregulated by Gal4. Transgenic flies having GFP or EP(3)3549 to express nep2 were crossed into flies expressing A-beta42, where expression of all the transgenes is under the control of UAS/GAL4. EP is located upstream of the nep2 gene in the same direction and is controlled by Gal4 expression. EP(3)3549 is a mutant without Gal4 because EP insertion can disrupt the endogenous gene expression of nep2 but could be upregulated by Gal4 because EP has Gal4 binding sites (UAS). Progeny flies co-expressing A-beta42 and nep2 or flies coexpressing A-beta42 and GFP were collected and allowed to age until they all died. The lifespans were compared.

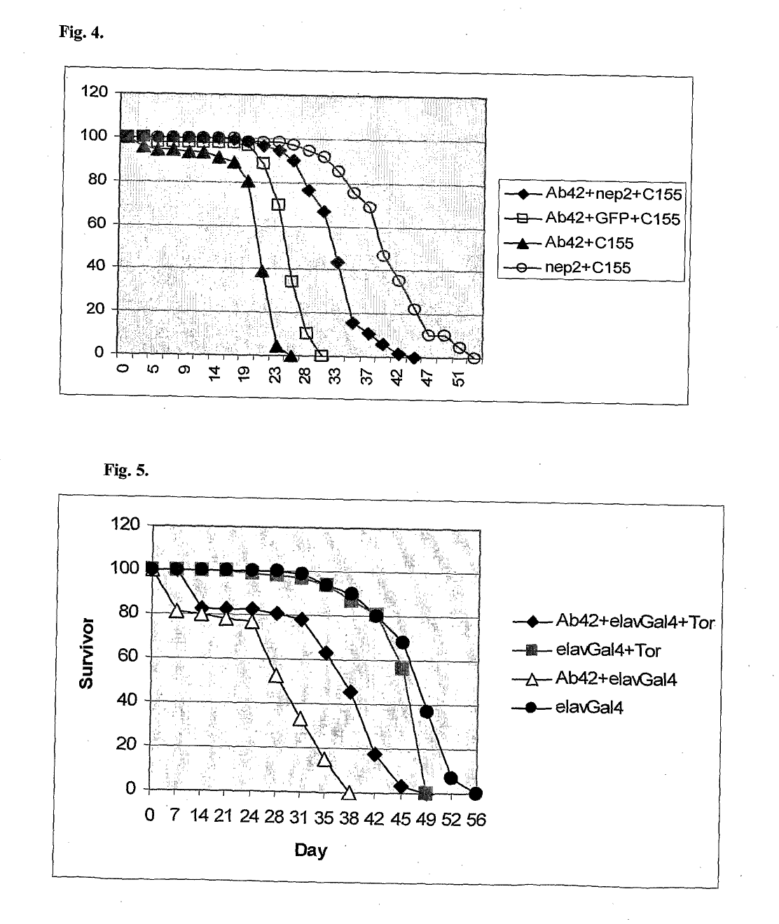

Flies co-expressing A-beta42 and nep2 (A-beta42+nep2+elavGal4) showed longer lifespan than those expressing A-beta42 (A-beta42+elavGal4) or co-expressing A-beta42 and GFP (A-beta42+GFP+elavGal4) (see FIG. 4); control flies expressing nep2 alone (nep2+elavGal4) had relatively normal lifespan. Thus, flies co-expressing A-beta42 and nep2 suppress the A-beta42-induced phenotype. It was also found that nep2 co-expression with A-beta42 leads to improved locomotion activity (Table 1), as compared to flies expressing A-beta42 and GFP, a non-toxic protein.

These results suggest that the interaction of A-beta42 and nep2 at the genetic level, and the subsequent suppression of the toxicity phenotype due to A-beta42, are not subject to cell type specificity.

### Example 6

### Further Analysis of Aβ42-Induced Rough Eye Phenotype Modifiers

We examined 23 previously identified mutations that can modify an A-beta42-induced rough eye phenotype, for their effects on lifespan and behavior phenotypes. Table 3 is the result of retesting EP lines for lifespan phenotype in flies expressing A-beta42 introduced by pan-neuronal Gal4, ElavGal4, in order to see whether genetic modification found from rough eye phenotype of KJ54 transgenic line (eye only phenotype) is the same genetic modification against lifespan phenotype of A-beta42 fly introduced by ElavGal4/UAS-A-beta42. It was found that their effects fell into 3 different classes. Eight mutations were found to modify the lifespan phenotypes in the same direction that they modified the eye phenotype, whereas nine modifiers of the eye phenotype had no effect on the lifespan phenotypes (Table 3), possibly because of differences in expression levels due to different Gal4 drivers. In the third class, 5 mutations had opposite modifying effects in the eye phenotype versus the lifespan phenotype (i.e. a suppressor of the eye phenotype acted as an enhancer of the lifespan phenotype). Finally, one EP mutation (EP(X)1318) did not give any viable progeny when co-expressed with A-beta42 (Table 3).

TABLE 3
Analysis of rough eye modifiers for modification of lifespan and behavioral phenotypes.

| | | | | | Modification |
|---|---|---|---|---|---|
| | | | | | of |
| | | | | Modification | locomotion |
| EP | | | Modification of | of lethal | phenotype |
| (Chr) | Gene/Annotation | Functions/Putative functional domains | eye phenotype | phenotype | (day 15) |
| EP(2) | escargot (CG3758), | RNA pol II transcription factor; acts as | Moderate E | Strong E | N/A |
| 0684 | GOF | transcriptional repressor; Expression in the | | (Developmental | |
| | | neurogenic region and antagonism of Scute | | lethal) | |
| | | and Daughterless suggest that escargot | | | |
| | | opposes a proneural fate; esg is a key | | | |
| | | regulator of cell adhesion | | | |
| EP(2) | ElbowB (CG4220)? | RNA polymerase II transcription factor, elB is | Moderate E | Moderate E | No effect |
| 0965 | | expressed in specific subset of tracheal cells | | | |
| | | and specifies distinct tracheal branching | | | |
| | | fates; ElB associates with Noc to form | | | |
| | | heterodimer; ElB may repress transcription of | | | |
| | | target genes involved in tracheal | | | |
| | | development | | | |
| EP(2) | P{EP}EP330 | Unknown | Mild/moderate E | Moderate E | No effect |
| 0330 | | | | | |
| EP(2) | CG7231; LOF (205 bp | Unknown | Mild E | Strong E | N/A |
| 2510 | from ATG within 5′ of | | | (Developmental | |
| | the gene on the | | | lethal) | |
| | opposite strand) | | | | |
| EP(3) | CG5490; Toll receptor | Tl receptor signalling pathway\|defense | mild E | mild E | No effect |
| 1051 | (FlyDB) No information | (immune) response | | | |
| | in Flybase; GOF (64 bp | | | | |
| | upstream to Tl) | | | | |
| EP(3) | 2 insertions depicted in | serine/threonine kinase (for EP(3)3015b) | Moderate E | Strong E | No effect |
| 3015 | Flybase; 3015a and b! | | | (Developmental | |
| | 3015a has no info; | | | lethal) | |
| | 3015b inserted within | | | | |
| | SNF4/AMP-activated | | | | |
| | protein kinase gamma | | | | |
| | subunit or | | | | |
| | SNF4Agamma | | | | |
| | (CG17299) gene (LOF | | | | |
| | or GOF?) | | | | |
| EP(3) | Neprilysin 2; GOF (93 bp | metallopeptidase | strong S | strong S | S |
| 3549 | upstream on the | | | | |
| | same strand) | | | | |
| EP(3) | gene CG10967 | serine/threonine kinase (CG10967) ribosomal | mild S | No effect | No effect |
| 3348 | (Flybase) CG11006 | protein (CG11006) | | | |
| | (FlyDB); LOF for | | | | |
| | CG10967 (100 bp within | | | | |
| | 5′ on opposite strand); | | | | |
| | GOF for CG11006 (961 bp | | | | |
| | upstream of it) | | | | |
| EP(3) | GOF for CG5517 (496 bp | CG5517 encodes Insulin degrading enzyme; | mild S | No effect | No effect |
| 3099 | upstream of it); LOF | CG5701 encodes RHO small GTPase | | | |
| | for CG5701 (77 bp | | | | |
| | within 5′ of this gene on | | | | |
| | opposite strand) | | | | |
| EP(X) | Unknown | Unknown | mild E | No effect | No effect |
| 1504 | | | | | |
| EP(X) | garnet (CG11197); LOF | encodes a product involved in ocellus | moderate S; | No effect | No effect |
| 0514 | (inserted 5.9 kb within | pigment biosynthesis | glassy eyes | | |
| | the gene on opposite | | without necrotic | | |
| | strand) | | spots in males; | | |
| | | | females have | | |
| | | | some necrotic | | |
| | | | spots | | |
| EP(X) | Silver (CG18503); LOF | fly carboxypeptidase Z homolog | mild E | mild E | E |
| 0356 | | | | | |
| EP(2) | Misexpression | Transcription factor; Zinc finger, C2H2 type, | Moderate E | Mild S | S |
| 0386 | suppressor of ras 4 | Elongation factor Ts (EF-Ts), dimerization | | | |
| | (CG4903); GOF (571 bp | domain | | | |
| | upstream) | | | | |
| EP(3) | CG6745 (LOF, sitting | CG6745 is Unknown; CG6765 is transcription | mild S | No effect | No effect |
| 0595 | within the gene at 3′ | factor | | | |
| | end, on same strand) or | | | | |
| | CG6765 (GOF, sitting | | | | |
| | 2.35 kb upstream to this | | | | |
| | gene) | | | | |
| EP(3) | FLYdb plot: Nearest | Unknown | likely mild S | No effect | E |
| 3041 | downstream gene on | | (semi lethal; | | |
| | the same strand is | | few exptal | | |
| | CG14959 at 16.3 kb | | males) | | |
| | (GOF?) | | | | |
| EP(3) | CG7437; mushroom- | It encodes a poly(rC) binding | mild S | No effect | E |
| 3108 | body expressed; LOF? | | | | |
| | (843 bp within 5′ of the | | | | |
| | gene on opposite | | | | |
| | strand) | | | | |
| EP(3) | CG6175 (FlyBase); | Unknown | mild S | Moderate E | E |
| 3405 | GOF (2 kb upstream of | | | | |
| | it) | | | | |
| EP(3) | Stich1 (CG17100) | RNA polymerase II transcription factor | mild S | No effect | No effect |
| 3470 | | | | | |
| EP(3) | LOF for CG6767 (1.1 kb | CG6767 encodes a ribose-phosphate | moderate S | mild E | No effect |
| 3603 | within 5′ of the gene on | pyrophosphokinase; CG8284 encodes | | | |
| | opposite strand) or GOF | ubiquitin conjugating enzyme | | | |
| | for CG8284 (2.7 kb | | | | |
| | upstream of it) | | | | |
| EP(X) | Dorsal switch protein 1 | it encodes a transcription co-repressor/single | mild S | No effect | No effect |
| 0355 | or DSP1(CG12223); | strand DNA binding | | | |
| | LOF? (13 bp within 5′ of | | | | |
| | the gene on the same | | | | |
| | strand)) | | | | |
| EP(X) | EG: 25E8.2 (CG2924); | It encodes an ubiquitin conjugating enzyme | moderate S | Mild E | E |
| 1596 | LOF (inserted 1.6 kb | involved in ubiquitin cycle | | | |
| | within the 5′ of gene on | | | | |
| | opposite strand) | | | | |
| EP(X) | LOF for CG1886 (4 bp | CG1886 encodes a copper exporting | mild S; eyes | Mild E | No effect |
| 0308 | upstream of this gene | ATPase; CG10347 has hsp-20 like | little rounder | | |
| | on the opposite strand) | chaperone domain | | | |
| | or GOF for CG10347 | | | | |
| | (304 upstream of it on | | | | |
| | the same strand) | | | | |
| EP(X) | Unknown | Unknown | mild E | cross doesn't | cross |
| 1318 | | | | work | doesn't |
| | | | | | work |

N/A, data not available.
E, enhancer;
S, suppressor (see Materials and Methods).
LOF, loss-of-function.
GOF, gain-of-function.

### Example 7

### HIGS to Find Genes that Modify the A-beta42 Over-Expression Dependent Lifespan Phenotype

A genetic screen using our model system to reveal novel genetic interactions that would enhance or suppress the A-beta42-induced lifespan phenotype was conducted. The screen utilizes a publicly-available collection of P-element insertion stocks in which the homozygous mutant is a developmental lethal (Spradling, A. C., Stern, D., Beaton, A., Rhem, E. J., Layerty, T., Mozden, N., Misra, S., and Rubin, G. M. (1999). The Berkeley Drosophila genome project gene disruption project. Single P-element insertions mutating 25% of vital Drosophila genes. Genetics 153: 135-177). These fly strains carry insertional mutations that cause reduction of gene function by generally interfering with transcription of the affected gene. This type of screen is called a haplo-insufficiency genetic screen (HIGS) since it relies on protein insufficiency arising from the presence of a mutation on one of the two alleles of a gene. In contrast to the EP type mutations, which in most cases cause upregulation of gene function, P-element mutations primarily cause “loss-of-function” of the affected gene. Transgenic adult flies expressing A-beta42 were crossed individually to 1,753 P-element fly strains and the progeny were scored for the changes in lifespan. After a primary screen, 152 enhancers and 185 suppressors were obtained.

One P-element was crossed into the transgenic A-beta42 expressing adult fly in order to generate desired progeny that carry A-beta42 expression and haplo-insufficiency of a gene linked to the P-element insertion (see FIG. 1). Therefore, it is expected to find a gene to ameliorate the A-beta42 induced toxic effect if the gene linked to the P-element modifies the toxic effect of A-beta42.

As seen in FIG. 1, the experimental progeny from these crosses carry copies of the A-beta-transgene on chromosome 2 and a copy of the P element on one of the sister chromosomes. Some P-elements were inserted to multiple place on genome so that there were more than two genes linked to a single P-element.

These progeny are compared to control progeny that have copies of A-beta-transgene on chromosome 2 but no P element on the sister chromosomes. The length of lifespan is compared between the experimental and control class of progeny. Any suppression or enhancement of lifespan caused by haplo-insufficiency of gene linked to P element is classified into a suppressor or an enhancer categories, respectively.

In order to confirm that the P-strains by themselves do not give an additive lifespan phenotype (or any other kind of eye defect), 103 suppressors were crossed to the flies carrying only the ElavGal4C¹⁵⁵ insertion on the X chromosome. In parallel to this genetic background check, re-screen crosses of the 103 suppressors and 58 enhancers were also performed with the transgenic A-beta42 expressing adult fly to confirm the results from the primary screen. These two sets of parallel experiments confirmed that 40 P-elements for suppressor and 21 P-elements for enhancer were reproducible (see Table 4). The 61 P-elements shown in Table 4 were exemplary sequences of identified genes affected by the mutations carried in the P-strains. Additional sequences which include variants of these genes and the proteins/polypeptides they encode, not shown here, were also included as additional targets covered by this invention.

TABLE 4
Annotation for 61 Modifier P-element Strains

| | | | | | Confirmed |
|---|---|---|---|---|---|
| P element | | Gene/ | | Functions/Putative functional | modifier of |
| No. | S/E | Annotation | CG No. | domains | A-beta42 |
| 11768 | S | Cross-bronx, | CG10536 | ubiquitin-conjugating enzymes; | Yes |
| | | cbx | | axon proning; a product involved in | |
| | | | | sperm individualization | |
| 11575 | S | Tramtrack | CG11558 | transcriptional repressor | Yes |
| 11533 | S | neuralized | CG11988 | ubiquitin-protein ligase | Yes |
| 10830 | S | l(2)k08816 | CG12744 | transcription regulator activity | Yes |
| | | | | putatively involved in nucleotide | |
| | | | | and nucleic acid metabolism | |
| 10536 | S | CAS/CSE1 | CG13281 | importin-alpha export receptor | Yes |
| | | segregation | | activity involved in protein-nucleus | |
| | | protein | | export; apoptosis | |
| 10691 | S | bancal, bl | CG13425 | RNA binding activity which was | Yes |
| | | | | localised to the cytoplasm and the | |
| | | | | nucleus; a KH domain | |
| 10691 | S | string | CG1395 | protein tyrosine/serine/threonine | Yes |
| | | | | phosphatase involved in G2/M | |
| | | | | transition of mitotic cell cycle | |
| 11191 | S | thickveins, | CG14026 | type I transforming growth factor- | Yes |
| | | tkv | | beta receptor activity, protein | |
| | | | | kinase; synaptic outgrowth | |
| 12085 | S | Ribosomal | CG17672 | protein biosynthesis; a ribosomal | Yes |
| | | protein S12, | | protein S12E and a ribosomal | |
| | | RpS12 | | protein L7AE | |
| 10536 | S | Midway | CG17938 | diacylglycerol O-acyltransferase | Yes |
| | | | | activity; triacylglycerol biosynthesis | |
| 12199 | S | Zinc finger | CG3260 | DNA binding, apoptosis; regulation | Yes |
| | | protein RP- | | of transcription from Pol II | |
| | | 8, Zfrp8 | | promoter | |
| 11757 | S | pelota, pelo | CG3959 | protein biosynthesis; meiotic cell | Yes |
| | | | | division; eRF1-like proteins, L30e- | |
| | | | | like | |
| 12177 | S | l(2)35Df | CG4152 | ATP dependent RNA helicase | Yes |
| 12219 | S | grapes, grp | CG4711 | serine/threonine kinase activity | Yes |
| | | | | involved in DNA damage response, | |
| | | | | signal transduction resulting in cell | |
| | | | | cycle arrest; cell cycle checkpoint; | |
| 10629 | | Cyclin- | CG5072 | cyclin-dependent protein kinase | Yes |
| | | dependent | | activity (EC: 2.7.1.—) | |
| | | kinase 4, | | serine/Threonine protein kinase | |
| | | Cdk4 | | family active site | |
| 11218 | S | Target of | CG5092 | phosphatidylinositol 3-kinase | Yes |
| | | rapamycin, | | (EC: 2.7.1.137) activity involved in | |
| | | Tor | | perception of nutrients | |
| 11533 | S | Na pump α | CG5670 | sodium/potassium-exchanging | Yes |
| | | subunit, | | ATPase activity (EC: 3.6.3.9); a | |
| | | Atpα | | na+, K+ ATPase β subunit, a | |
| | | | | H+/K+ and Na+/K+ transporting | |
| | | | | ATPase | |
| 11726 | S | l(3)07882 | CG5824 | Homo sapiens ‘unknown protein | Yes |
| | | | | IT12’ DYNACTIN 1-RELATED | |
| 15174 | S | CG6218 | CG6218 | carbohydrate kinase activity | Yes |
| | | | | putatively involved in carbohydrate | |
| | | | | metabolism | |
| 16301 | S | Smg6 | CG6369 | mRNA catabolism, nonsense- | Yes |
| | | | | mediated; Tetratricopeptide repeat | |
| | | | | (TPR), | |
| 11175 | S | Connector | CG6556 | enzyme binding involved in torso | Yes |
| | | enhancer of | | signaling pathway to the cell-cell | |
| | | ksr, cnk | | adherens junction; PDZ domain | |
| | | | | (aka, DHR or GLGF) | |
| 12422 | S | CG7878 | CG7878 | a product with ATP dependent | Yes |
| | | | | RNA helicase activity (EC: 3.6.1.3) | |
| 10935 | S | Elongation | CG8280 | a GTP-binding elongation factor | Yes |
| | | factor | | | |
| | | 1α48D, | | | |
| | | Ef1α48D | | | |
| 10557 | S | Heat shock | CG8542 | Heat shock protein 70 kD (HSP70), | Yes |
| | | protein | | C-terminal, substrate-binding | |
| | | cognate 5, | | fragment, | |
| | | Hsc70-5 | | | |
| 16101 | S | Guanyl | CG8742 | guanylate cyclase; receptor activity | Yes |
| | | cyclase at | | | |
| | | 76C, | | | |
| | | Gyc76C | | | |
| 11371 | S | Naca | CG8759 | Nascent polypeptide associated | Yes |
| | | | | complex protein alpha subunit; TS- | |
| | | | | N domain | |
| 12382 | S | wunen, | CG8804 | phosphatidate phosphatase activity; | Yes |
| | | wun | | G-protein coupled receptor protein | |
| | | | | signaling pathway; lipid metabolism | |
| 10178 | S | 26-29 kD- | CG8947 | cathepsin K activity; proteolysis and | Yes |
| | | proteinase | | peptidolysis; insensitive to specific | |
| | | | | inhibitors of cathepsin L | |
| 10673 | S | l(2)k07502b | CG9523 | Putative tetratricopetide repeat (or | Yes |
| | | | | putative huntingtin interacting | |
| | | | | protein) | |
| 11595 | S | Tryptophan | CG9735 | a product with tryptophan-tRNA | Yes |
| | | yl-tRNA | | ligase activity (EC: 6.1.1.2) involved | |
| | | synthetase, | | in tryptophanyl-tRNA | |
| | | Aats-trp | | aminoacylation | |
| 12379 | S | Decapentap | CG9885 | TGFbeta receptor ligand; Inhibin α | Yes |
| | | legic, dpp | | chain, Cystine-knot cytokines, | |
| 10197 | S | skuld, skd | CG9936 | RNA polymerase II transcription | Yes |
| | | | | mediator; Suppressor of | |
| | | | | constitutively activated Dpp | |
| | | | | signaling 78 | |
| 12382 | S | l(2)k10201 | CG13951 | Zinc finger, C2H2 type | Yes |
| 12174 | S | lethal(2) | CG4180 | Unknown | Yes |
| | | 35Bg | | | |
| 10839 | S | l(2)44Db | CG14750 | Unknown | Yes |
| 12411 | S | CG11376 | CG11376 | Unknown | Yes |
| 16053 | S | CG1142 | CG1142 | Unknown | Yes |
| 12209 | S | l(2)k03704 | CG3776 | Unknown | Yes |
| 12392 | S | l(2)10333 | unknown | Unknown | Yes |
| 12555 | S | P{GT1}BG01022 | unknown | Unknown | Yes |
| 10941 | S | l(2)00572 | unknown | Unknown | Yes |
| 11577 | S | l(3)02732 | unknown | Unknown | Yes |
| 11585 | S | l(3)03342 | unknown | Unknown | Yes |
| 12184 | S | l(2)k07521 | unknown | Unknown | Yes |
| 12372 | S | l(2)10491 | unknown | Unknown | Yes |
| 10523 | E | Burgundy, | CG9242 | GMP synthase (glutamine | Yes |
| | | bur | | hydrolyzing) activity putatively | |
| | | | | involved in GMP biosynthesis; a | |
| | | | | glutamine amidotransferase class-I, | |
| | | | | a carbamoyl-phosphate synthase, | |
| | | | | GATase domain and a GMP | |
| | | | | synthase C terminal domain. | |
| 10534 | E | mitochondrial | CG5012 | structural constituent of ribosome | Yes |
| | | ribosomal | | involved in protein biosynthesis | |
| | | protein | | which was a component of the | |
| | | L7/L12, | | mitochondrial large ribosomal | |
| | | mRpL7-L12 | | subunit; a ribosomal protein L7/L12 | |
| | | | | C-terminal domain | |
| 10615 | E | par-1 | CG11960 | a product with protein | Yes |
| | | | | serine/threonine kinase activity | |
| | | | | involved in oocyte microtubule | |
| | | | | cytoskeleton organization which | |
| | | | | was a component of the cell cortex; | |
| | | | | an eukaryotic protein kinase, a | |
| | | | | tyrosine kinase catalytic domain | |
| 10646 | E | split ends, | CG18497 | a product involved in glia cell | Yes |
| | | spen | | migration which was localised to | |
| | | | | the nucleus; RNA-binding region | |
| | | | | RNP-1 (RNA recognition motif) | |
| 11031 | E | heixuedian, | CG5876 | a product which was putatively a | Yes |
| | | heix | | component of the integral to | |
| | | | | membrane; an ubiA | |
| | | | | prenyltransferase | |
| 10624 | E | CG13438 | CG13438 | Unknown | Yes |
| 11032 | E | l(2)k11404 | unknown | Unknown | Yes |
| 11036 | E | l(2)k11405 | unknown | Unknown | Yes |
| 11145 | E | l(2)k15617 | unknown | Unknown | Yes |
| 10493 | E | l(2)k01206 | unknown | Unknown | Yes |
| 10510 | E | l(2)k02205 | unknown | Unknown | Yes |
| 10511 | E | l(2)k02206 | unknown | Unknown | Yes |
| 10523 | E | l(2)k03107 | unknown | Unknown | Yes |
| 10533 | E | l(2)k03609 | unknown | Unknown | Yes |
| 10534 | E | l(2)k03610 | unknown | Unknown | Yes |
| 10538 | E | l(2)k04003 | unknown | Unknown | Yes |
| 10578 | E | l(2)k05812 | unknown | Unknown | Yes |
| 10869 | E | l(2)k09221 | unknown | Unknown | Yes |
| 10964 | E | l(2)k10004 | unknown | Unknown | Yes |
| 10968 | E | l(2)k10105 | unknown | Unknown | Yes |
| 10973 | E | l(2)k10127 | unknown | Unknown | Yes |
| 10985 | E | l(2)k16510 | unknown | Unknown | Yes |
| 11005 | E | l(2)k10815 | unknown | Unknown | Yes |

S = Suppressor;
E = Enhancer (see Materials and Methods).
CG No. designations are from Flybase (http://flybase.bio.indiana.edu/) and from FLyDB3 and FLyDB4.

### Example 8

### Human Homologues of the 61 P-Element Modifiers

In parallel to ongoing validation assays for the 61 A-beta42-modifiers in the Drosophila system, bioinformatics analysis was used to identify the human homologues/orthologues of the fly genes affected by these modifier P-insertions. The insertion site for 24 P-modifiers (12392, 12555, 10941, 11577, 11585, 12184, 12372, 11032, 11036, 11145, 10493, 10510, 10511, 10523, 10533, 10534, 10538, 10578, 10869, 10964, 10968, 10973, 10985, and 11005) was not available and thus the affected gene in these strains was unknown and accompanied by unknown CG number.

To analyze the nature of the mutations caused by each P-insertion, information was gathered from Flybase, a public database (http://flybase.net/), and FlyDB3 and FlyDB4, proprietary databases, in order to find the genes mutated by the insertion. Based on the relative distance from the insertion and the orientation, the genes that can possibly be affected by the P-insertion were identified and used for BLAST analysis of human genes. Thus in the BLAST searches the query sequences were fly CG No. sequences obtained from Flybase, FlyDB3 and FlyDB4, and the matched sequences found are human sequences showing homology to the fly query sequence. Furthermore, one P-element insertion can disrupt two genes when the two genes are close to the insertion and/or they share the same genomic location regardless of whether the gene expression direction of the two genes is opposite or same.

Forty-four (44) genes were found to be in the vicinity of the 37 P-elements, so that they could potentially be affected by the P-insertions. These 44 genes were subjected to BLAST analysis according to conventional methods. Some Drosophila genes have no human ortholog as there has been a gene duplication in the diverging lineages between fly and human. Many neural genes have duplicated in human and there was only one copy of the gene in Drosophila. So in these cases there was no corresponding human ortholog. Parameters for the mapping of the Drosophila melanogaster protein sequences to Refseq, Celera and Compugen protein sequences were as follows: The e-value cutoff was 1e-10, with the strong constraint of mutual best pairwise BLAST match between the two genomes. Most of the expectation values corresponded to much greater significance than this cutoff value. Refseq release April 2002 and Celera proteins R26j were used as the databases for the BLAST analysis. Homologous sequences were found for 31 of the 44 genes.

Annotations were found for 31 of the 44 human orthologs in the Celera database (see Table 5). Thirty one (31) of the human homologues were then analyzed by reverse BLAST analysis back to the Drosophila protein database. This analysis provided the original Drosophila sequences as the matches, indicating that they were orthologues to the human sequences. This supports the validity of the approach described here.

The identified suppressors are genes whose annotations correspond to a variety of molecular or physiological functions, indicating that there may be several ways to ameliorate A-beta42 induced toxicity. For example, a subset of genes falls into the dpp-tkv signaling pathway (dpp, tkv, and skd), while others appear to be involved in regulation of the cell cycle. Some suppressor genes have previously identified involvement in Alzheimer's disease or other neurodegerative diseases such as Huntington's disease (polyglutamine repeat pathology), while other genes were novel, and may play a previously unrecognized role in neurodegeneration revealed by the Drosophila model for AD. Full-length sequences (see the Sequence Listing) were available for 31 of the available human orthologues (see Table 6).

TABLE 5
Annotations for the Human ortholog of the 36 modifiers

| Drosophila | | | | | |
|---|---|---|---|---|---|
| melanogaster | | Homo Sapiens | | | |
| P | | | Best matching | | |
| element | Drosophila | Refseq | Celera human | | |
| No. | gene ID | protein ID | protein | Celera gene | Description/Annotation |
| 10178 | CG8947 | NP_000387.1 | hCP1859254 | hCG39377 | cathepsin K (pycnodysostosis) |
| 10197 | CG9936 | | hCP45795.2 | hCG32573 | thyroid hormone receptor-associated protein, 240 kDa subunit |
| 10536 | CG31991-PA | NP_036211.1 | hCP1872018 | hCG24006 | diacylglycerol O-acyltransferase 1; acyl coenzyme |
| | | | | | A: cholesterol acyltransferase related gene 1; diglyceride |
| | | | | | acyltransferase; ACAT related gene product 1 |
| 10536 | CG13281-PA | NP_001307.2 | hCP1857430 | hCG2019736 | CSE1 chromosome segregation 1-like protein isoform a; |
| | | | | | cellular apoptosis susceptibility protein; importin-alpha re- |
| | | | | | exporter |
| 10557 | CG8542-PA | NP_004125.3 | hCP1762395.1 | hCG18779 | heat shock 70 kDa protein 9B precursor; heat shock 70 kD |
| | | | | | protein 9; stress-70 protein, mitochondrial; 75 kDa glucose |
| | | | | | regulated protein; peptide-binding protein 74; mortalin, |
| | | | | | perinuclear; p66-mortalin |
| 10629 | CG5072-PA | NP_001250.1 | hCP1873749 | hCG19542 | (NM_001259) cyclin-dependent kinase 6; cell division protein |
| | | | | | kinase 6 |
| 10691 | CG13425-PB | NP_002131.2 | hCP1804927 | hCG1985922 | heterogeneous nuclear ribonucleoprotein K isoform a; dC- |
| | | | | | stretch binding protein; transformation upregulated nuclear |
| | | | | | protein |
| 10839 | CG14750-PA | NP_115729.1 | hCP37242.1 | hCG16964 | (NM_032353) hypothetical protein MGC10540 |
| 10935 | CG8280 | | hCP1877457 | hCG2033271 | eukaryotic translation elongation factor 1 alpha 1 |
| 11191 | CG14026-PA | NP_001194.1 | hCP49917.2 | hCG38702 | bone morphogenetic protein receptor, type IB; |
| | | | | | serine/threonine receptor kinase |
| 11218 | CG5092-PA | NP_004949.1 | hCP1794677 | hCG24781 | FK506 binding protein 12-rapamycin associated protein 1; |
| | | | | | FK506 binding protein 12-rapamycin associated protein 2; |
| | | | | | rapamycin target protein; FKBP12-rapamycin complex- |
| | | | | | associated protein 1; FKBP-rapamycin associated protein |
| 11371 | CG8759-PA | NP_005585.1 | hCP1880630 | hCG2016482 | nascent-polypeptide-associated complex alpha polypeptide |
| 11533 | CG1395 | NP_068660.1 | hCP1862937 | hCG39252 | cell division cycle 25B |
| | | NP_068658.1 | | | |
| 11533 | CG5670 | NP_000692.2 | hCP39434.2 | hCG37943 | ATPase, Na+/K+ transporting, alpha 1 polypeptide |
| 11595 | CG9735 | NP_776049.1 | hCP1867986 | hCG25017 | tryptophanyl-tRNA synthetase |
| 11726 | CG5824 | | hCP1865495 | hCG20634 | |
| 11768 | CG10536 | NP_071921.1 | hCP1878192 | hCG2025804 | fused toes homolog (mouse) |
| 12085 | CG17672 | NP_001007.2 | hCP1876196 | hCG2030596 | ribosomal protein S12 |
| 12174 | CG4180 | | hCP1774435 | hCG16498 | hypothetical protein LOC57019 |
| 12177 | CG4152 | NP_795714.1 | hCP1814106 | hCG40991 | KIAA0052 protein |
| 12199 | CG3260 | NP_002589.2 | hCP1875254 | hCG2029505 | programmed cell death 2 |
| 12372 | CG6556 | | hCP1898224 | hCG18974 | connector enhancer of KSR2 |
| 12382 | CG8804 | | hCP1767970 | hCG39580 | phosphatidic acid phosphatase type 2A |
| 12411 | CG11376-PA | NP_065863.1 | hCP44734.3 | hCG29966 | dedicator of cytokinesis 6 |
| 12422 | CG7878-PA | NP_061135.1 | hCP40327.2 | hCG22906 | DEAD-box protein |
| 16053 | CG1142 | | hCP45488.2 | hCG31941 | acidic 82 kDa protein mRNA |
| 16101 | CG8742 | | hCP201087.2 | hCG96602.2 | natriuretic peptide receptor A/guanylate cyclase A |
| | | | | | (atrionatriuretic peptide receptor A) |
| 16301 | CG6369 | | hCP1890713.1 | hCG1813980 | chromosome 17 open reading frame 31 |
| 11575 | CG11558 | No ortholog found | | | |
| 12219 | CG4711 | No ortholog found | | | |
| 10534 | CG5012-PA | NP_002940.2 | hCP1789470 | hCG2040013 | mitochondrial ribosomal protein L12 |
| 10523 | CG9242-PA | NP_003866.1 | hCP1761705.1 | hCG1811302 | guanine monophosphate synthetase; guanosine 5′- |
| | | | | | monophosphate synthase; glutamine amidotransferase; GMP |
| | | | | | synthase |
| 11031 | CG5876-PA | NP_037451.1 | hCP41828.1 | hCG24775 | transitional epithelia response protein |

| | | |
|---|---|---|
| 11960 | CG11960 | No ortholog found |
| | (CG30131-PA) | |
| 10624 | CG13438 | No ortholog found |
| 10615 | CG18497 | No ortholog found |

TABLE 6
Human orthologues to 31 Drosophila modifiers

| Drosophila melanogaster | | | Homo Sapiens | | | |
|---|---|---|---|---|---|---|
| | Drosophila | | | Best matching | | |
| | gene | Modification | Refseq | Celera human | | SEQ ID |
| Modifier | ID | S/E | protein ID | protein | Celera gene | NO: |
| 10178 | CG8947 | S | NP_000387.1 | hCP1859254 | hCG39377 | 1 |
| 10197 | CG9936 | S | | hCP45795.2 | hCG32573 | 2 |
| 10536 | CG31991-PA | S | NP_036211.1 | hCP1872018 | hCG24006 | 3 |
| 10536 | CG13281-PA | S | NP_001307.2 | hCP1857430 | hCG2019736 | 4 |
| 10557 | CG8542-PA | S | NP_004125.3 | hCP1762395.1 | hCG18779 | 5 |
| 10629 | CG5072-PA | S | NP_001250.1 | hCP1873749 | hCG19542 | 6 |
| 10691 | CG13425-PB | S | NP_002131.2 | hCP1804927 | hCG1985922 | 7 |
| 10839 | CG14750-PA | S | NP_115729.1 | hCP37242.1 | hCG16964 | 8 |
| 10935 | CG8280 | S | | hCP1877457 | hCG2033271 | 9 |
| 11191 | CG14026-PA | S | NP_001194.1 | hCP49917.2 | hCG38702 | 10 |
| 11218 | CG5092-PA | S | NP_004949.1 | hCP1794677 | hCG24781 | 11 |
| 11371 | CG8759-PA | S | NP_005585.1 | hCP1880630 | hCG2016482 | 12 |
| 11533 | CG1395 | S | NP_068660.1 | hCP1862937 | hCG39252 | 13 |
| | | | NP_068658.1 | | | |
| 11533 | CG5670 | S | NP_000692.2 | hCP39434.2 | hCG37943 | 14 |
| 11595 | CG9735 | S | NP_776049.1 | hCP1867986 | hCG25017 | 15 |
| 11726 | CG5824 | S | | hCP1865495 | hCG20634 | 16 |
| 11768 | CG10536 | S | NP_071921.1 | hCP1878192 | hCG2025804 | 17 |
| 12085 | CG17672 | S | NP_001007.2 | hCP1876196 | hCG2030596 | 18 |
| 12174 | CG4180 | S | | hCP1774435 | hCG16498 | 19 |
| 12177 | CG4152 | S | NP_795714.1 | hCP1814106 | hCG40991 | 20 |
| 12199 | CG3260 | S | NP_002589.2 | hCP1875254 | hCG2029505 | 21 |
| 12372 | CG6556 | S | | hCP1898224 | hCG18974 | 22 |
| 12382 | CG8804 | S | | hCP1767970 | hCG39580 | 23 |
| 12411 | CG11376-PA | S | NP_065863.1 | hCP44734.3 | hCG29966 | 24 |
| 12422 | CG7878-PA | S | NP_061135.1 | hCP40327.2 | hCG22906 | 25 |
| 16053 | CG1142 | S | | hCP45488.2 | hCG31941 | 26 |
| 16101 | CG8742 | S | | hCP201087.2 | hCG96602.2 | 27 |
| 16301 | CG6369 | S | | hCP1890713.1 | hCG1813980 | 28 |
| 10534 | CG5012-PA | E | NP_002940.2 | hCP1789470 | hCG2040013 | 29 |
| 10523 | CG9242-PA | E | NP_003866.1 | hCP1761705.1 | hCG1811302 | 30 |
| 11031 | CG5876-PA | E | NP_037451.1 | hCP41828.1 | hCG24775 | 31 |

S = Suppressor;
E = Enhancer (see Materials and Methods).

### Example 9

### Haplo-Insufficiency Analysis of Drosophila Target of Rapamycin (dTor)

From Table 4, the Drosophila Target of rapamycin (Tor, or dTor, herein) (P element No. 11218, CG5092) was chosen for follow up because of the availability of mutations and compounds that effect Tor function. Lifespan was then scored for experimental flies which expressing A-beta42 in all neurons and bearing one copy of P-element 11218 insertion or one independent mutant copy of dTor^{ΔP} that is allelic to P-element 11218. Lifespan and behavior of flies heterozygous for dTor that express A-beta42 was compared to those of flies that carry driver only and one mutant copy of dTor. Lifespan was scored until all flies died. The climbing assay was performed at 21 days to assess any improvement of locomotor activity. The fly brains were dissected out at 21 days to see vacuolization.

From FIG. 5 it is seen that flies expressing A-beta42 and having one mutant copy of Tor gene (Ab42+elavGal4+Tor) exhibit extended lifespan compared to flies expressing A-beta42 alone (Ab42+elavGal4). Control flies expressing the Tor mutation alone (elavGal4+Tor) or the driver alone (elavGal4) had normally long lifespans.

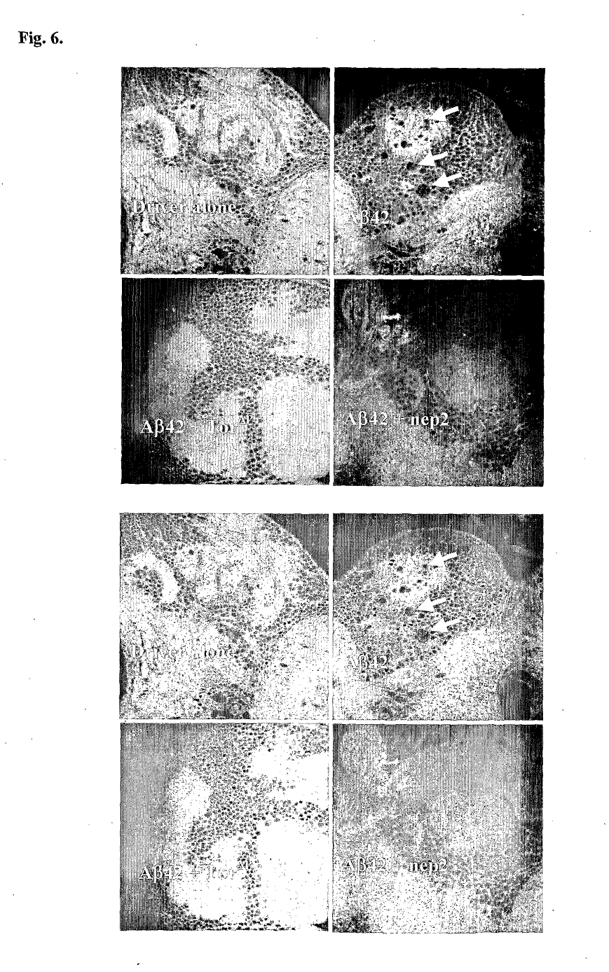

Brains of 21 day old flies were dissected and stained with NCB C₆-ceramide for neuropil (Green) and propidium iodide (PI) for nueclei (Red). Four brains of each genotype were analyzed. Images are shown in FIG. 6. Brain expressing A-beta42 in all neurons has vacuoles (or holes) indicated by arrows (A-beta42, upper right panel). No holes were found in the driver alone control (upper left panel). Brain from flies expressing A-beta42 and carrying a mutant copy of Tor shows no vacuoles (AP42+Tor^{ΔP}, lower left panel). These results suggests that reduced activity of Tor function, achieved by introducing a mutant copy of Tor into flies expressing A-beta42, rescues the vacuole phenotype. Brain from flies expressing A-beta42 and nep2 likewise shows no vacuoles (Aβ42+nep2, lower right panel).

These results indicate that partial inhibition of Tor function by introduction of one mutant copy of the dTor gene into flies expressing A-beta42, using P-element 11218, and the independent allele, dTor^{ΔP}; ameliorates A-beta42 mediated toxicity. This manipulation partially rescues A-beta42 induced shortened lifespan, vacuoles in the brain, and the locomotor deficit (data not shown).

### Example 10

### Treatment of Flies Expressing A-beta42 with an Inhibitor of Target of Rapamycin

Genetic reduction of Tor function by haplo-insufficiency (−/+) shows partial rescue of lifespan, locomotor, and CNS vacuolization phenotypes. A proprietary inhibitor of mTor (mammalian Tor), Novartis compound RAD001, was assessed for its ability to inhibit endogenous Tor function in Drosophila. The structure of RAD001 is:

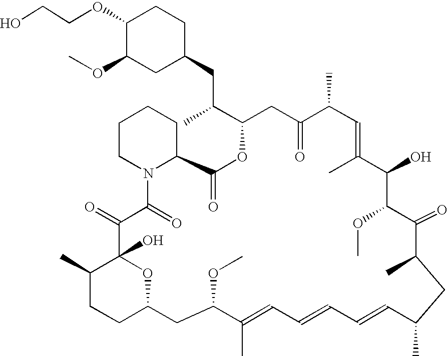

If successful, this treatment would provide the same beneficiary effect on A-beta42 induced toxicity found through genetic modification. Flies expressing A-beta42 were fed every three days with 10 uM, 20 uM, 30 uM and 60 uM (where uM=10⁻⁶ M) concentration of RAD001. Lifespan was scored until all the RAD001 fed flies died. The climbing assay was performed on RAD001 fed flies at 21 day, and compared to placebo fed flies.

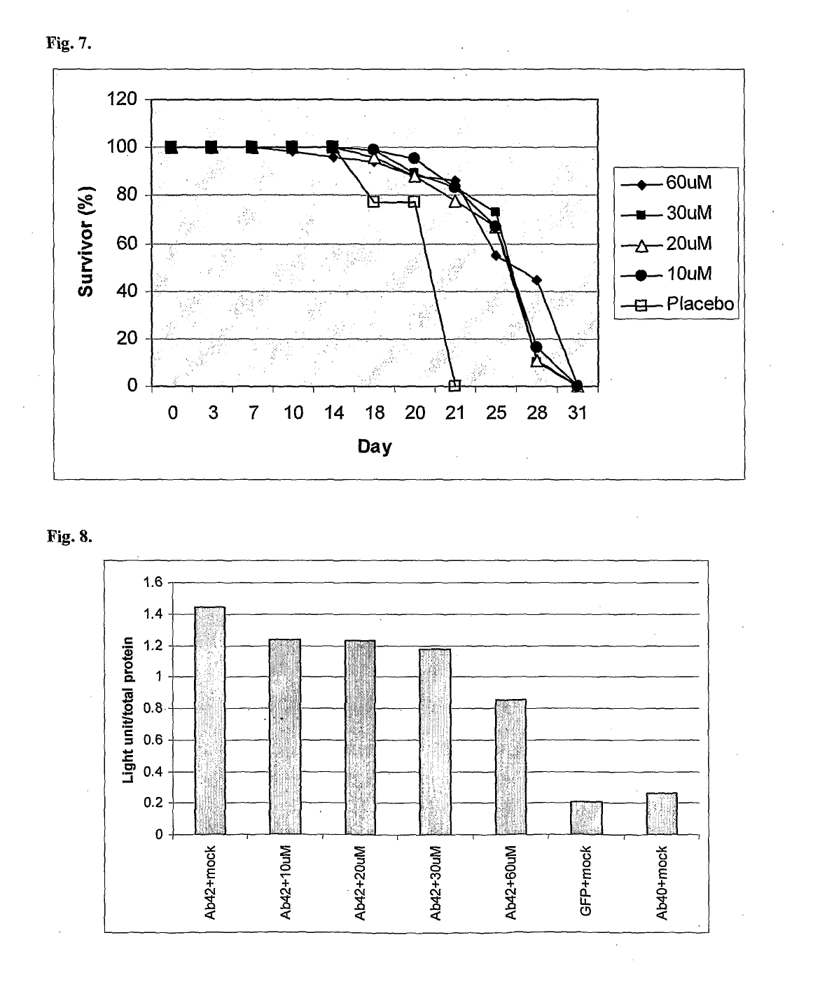

We found that RAD001 treatment of transgenic flies expressing A-beta42 results in partial rescue of lifespan, even at the lowest concentration of RAD001 tested (see FIG. 7). The locomotor defect was also partially abrogated (data not shown) compared to placebo fed flies. These data indicate that inhibition of Tor by RAD001 ameliorates the toxic effect of A-beta42 in transgenic flies. These results complement the results in Example BC due to the partial loss of Tor function induced by haplo-insufficiency.

### Example 11

### Haplo-Insufficiency of Other Genes Potentially Affecting the A-beta42-Induced Lifespan Phenotype

Since normal Tor function is implicated in nutrition sensing (the Tor pathway) and is linked to the insulin signaling pathway, mutant alleles of other key genes in the Tor pathway, upstream or downstream effectors such as TSC1/2, S6K and eIF-4B, and alleles such as InR, GSK and PTEN in the insulin signaling pathway were examined to test for haplo-insufficient (+/−) modification of the A-beta42-induced lifespan phenotype. With the exception of TSC2, which acted as a mild suppressor, none of the other Tor pathway-associated genes and insulin signaling pathway-associated genes modified the lifespan phenotype in this experimental condition.

### Example 12

### Analysis of the Amount of A-beta42 Extracted from Transgenic Flies Treated with RAD001

The amount of A-beta42 from the heads of aged flies expressing A-beta42 was measured to see whether inactivation or reduced activation of Tor function affects the amount of A-beta42, such that it would correlate with the amelioration of A-beta42 induced toxicity. 20 heads from 13 day old flies expressing A-beta42 and fed 10 uM, 20 uM, 30 uM, or 60 uM RAD001 every three days, or fed no RAD001, as well as control transgenic flies, were ground in ELISA buffer (150 mM NaCl, 0.5% IGEPAL® CA-630, 0.05% sodium deoxycholate, and 50 mM Tris, pH 8.0), which allows to extract total A-beta42, and the amount of A-beta42 was measured by sandwich ELISA.

FIG. 8 shows that flies fed RAD001 exhibit reduction of the amount of soluble A-beta42 (up to 42%). The figure provides the amounts of A-beta42 found in each experiment after normalization of the A-beta42 to total protein. Flies expressing A-beta42 that were fed placebo show a higher amount of A-beta42 than flies fed RAD001. The flies dosed with 60 uM RAD001 exhibit a pronounced decrease of total A-beta42. Negative control flies expressing A-beta40 and fed placebo exhibit a minimal assay level, which is identical to the negative control of transgenic flies expressing GFP and fed placebo. These data suggest that RAD001 treatment inhibits the endogenous dTor activity, and acts to deplete A-beta42 and as a result reduces total A-beta42 from the heads of the flies. Similar results were observed from flies fed RAD001 every day and every 2 days.

Without wishing to be bound by theory, a possible mechanism for A-beta42 clearance through the Tor signaling pathway is likely through autophagy, because Tor negatively regulates autophagy 1 (ATG1) protein through phosphorylation. This mechanism has recently been shown to operate in Tor mediated suppression of polyglutamine repeat neurotoxicity (See Ravikumar et al, Nat Genet, Vol. 36, No. 6, 585-595 (2004)).

### Protein Sequence Listings

SEQ ID NO: 1

MWGLKVLLLPVVSFALYPEEILDTHWELWKKTHRKQYNNKVDEISRRLIW
EKNLKYISIHNLEASLGVHTYELAMNHLGDMTSEEVVQKMTGLKVPLSHS
RSNDTLYIPEWEGRAPDSVDYRKKGYVTPVKNQGQCGSCWAFSSVGALEG
QLKKKTGKLLNLSPQNLVDCVSENDGCGGGYMTNAFQYVQKNRGIDSEDA
YPYVGQEESCMYNPTGKAAKCRGYREIPEGNEKALKRAVARVGPVSVAID
ASLTSFQFYSKGVYYDESCNSDNLNHAVLAVGYGIQKGNKHWIIKNSWGE
NWGNKGYILMARNKNNACGIANLASFPKM*

SEQ ID NO: 2

MSASFVPNGASLEDCHCNLFCLADLTGIKWKKYVWQGPTSAPILFPVTEE
DPILSSFSRCLKADVLGVWRRDQRPGRRELWIFWWGEDPSFADLIHHDLS
EEEDGVWENGLSYECRTLLFKAVHNLLERCLMNRNFVRIGKWFVKPYEKD
EKPINKSEHLSCSFTFFLHGDSNVCTSVEINQHQPVYLLSEEHITLAQQS
NSPFQVILCPFGLNGTLTGQAFKMSDSATKKLIGEWKQFYPISCCLKEMS
EEKQEDMDWEDDSLAAVEVLVAGVRMIYPACFVLVPQSDIPTPSPVGSTH
CSSSCLGVHQVPASTRDPAMSSVTLTPPTSPEEVQTVDPQSVQKWVKFSS
VSDGFNSDSTSHHGGKIPRKLANHVVDRVWQECNMNRAQNKRKYSASSGG
LCEEATAAKVASWDFVEATQRTNCSCLRHKNLKSRNAGQQGQAPSLGQQQ
QILPKHKTNEKQEKSEKPQKRPLTPFHHRVSVSDDVGMDADSASQRLVIS
APDSQVRFSNIRTNDVAKTPQMHGTEMANSPQPPPLSPHPCDVVDEGVTK
TPSTPQSQHFYQMPTPDPLVPSKPMEDRIDSLSQSFPPQYQEAVEPTVYV
GTAVNLEEDEANIAWKYYKFPKKKDVEFLPPQLPSDKFKDDPVGPFGQES
VTSVTELMVQCKKPLKVSDELVQQYQIKNQCLSAIASDAEQEPKIDPYAF
VEGDEEFLFPDKKDRQNSEREAGKKHKVEDGTSSVTVLSHEEDAMSLFSP
SIKQDAPRPTSHARPPSTSLIYDSDLAVSYTDLDNLFNSDEDELTPGSKK
SANGSDDKASCKESKTGNLDPLSCISTADLHKMYPTPPSLEQHIMGFSPM
NMNNKEYGSMDTTPGGTVLEGNSSSIGAQFKIEVDEGFCSPKPSEIKDFS
YVYKPENCQILVGCSMFAPLKTLPSQYLPPIKLPEECIYRQSWTVGKLEL
LSSGPSMPFIKEGDGSNMDQEYGTAYTPQTHTSFGMPPSSAPPSNSGAGI
LPSPSTPRFPTPRTPRTPRTPRGAGGPASAQGSVKYENSDLYSPASTPST
CRPLNSVEPATVPSIPEAHSLYVNLILSESVMNLFKDCNFDSCCICVCNM
NIKGADVGVYIPDPTQEAQYRCTCGFSAVMNRKFGNNSGLFLEDELDIIG
RNTDCGKEAEKRFEALRATSAEHVNGGLKESEKLSDDLILLLQDQCTNLF
SPFGAADQDPFPKSGVISNWVRVEERDCCNDCYLALEHGRQFMDNMSGGK
VDEALVKSSCLHPWSKRNDVSMQCSQDILRMLLSLQPVLQDAIQKKRTVR
PWGVQGPLTWQQFHKMAGRGSYGTDESPEPLPIPTFLLGYDYDYLVLSPF
ALPYWERLMLEPYGSQRDIAYVVLCPENEALLNGAKSFFRDLTAIYESCR
LGQHRPVSRLLTDGIMRVGSTASKKLSEKLVAEWFSQAADGNNEAFSKLK
LYAQVCRYDLGPYLASLPLDSSLLSQPNLVAPTSQSLITPPQMTNTGNAN
TPSATLASAASSTNTVTSGVAISTSVATANSTLTTASTSSSSSSNLNSGV
SSNKLPSFPPFGSMNSNAAGSMSTQANTVQSGQLGGQQTSALQTAGISGE
SSSLPTQPHPDVSESTMDRDKVGIPTDGDSHAVTYPPAIVVYIIDPFTYE
NTDESTNSSSVWTLGLLRCFLEMVQTLPPHIKSTVSVQIIPCQYLLQPVK
HEDREIYPQHLKSLAFSAFTQCRRPLPTSTNVKTLTGFGPGLAMETALRS
PDRPECIRLYAPPFILAPVKDKQTELGETFGEAGQKYNVLFVGYCLSRDQ
RWILASCTDLYGELLETCIINIDVPNRARRKKSSARKFGLQKLWEWCLGL
VQMSSLPWRVVIGRLGRIGHGELKDWSCLLSRRNLQSLSKRLKDMCRMCG
ISAADSPSILSACLVAMEPQGSFVIMPDSVSTGSVFGRSTTLNMQTSQLN
TPQDTSCTHILVFPTSASVQVASATYTTENLDLAFNPNNDGADGMGIFDL
LDTGDDLDPDIINILPASPTGSPVHSPGSHYPHGGDAGKGQSTDRLLSTE
PHEEVPNILQQPLALGYFVSTAKAGPLPDWFWSACPQAQYQCPLFLKASL
HLEVPSVQSDELLHSKHSHPLDSNQTSDVLRFVLEQYNALSWLTCDPATQ
DRRSCLPIHFVVLNQLYNFINNML*

SEQ ID NO: 3

MGDRGSSRRRRTGSRPSSHGGGGPAAAEEEVRDAAAGPDVGAAGDAPAPA
PNKDGDAGVGSGHWELRCHRLQDSLFSSDSGFSNYRGILNWCVVMLILSN
ARLFLENLIKYGILVDPIQVVSLFLKDPYSWPAPCLVIAANVFAVAAFQV
EKRLAVGALTEQAGLLLHVANLATILCFPARVVLLVESITPVGSLLALMA
HTILFLKLFSYRDVNSWCRRARAKAASAGKKASSAAAPHTVSYPDNLTYR
DLYYFLFAPTLCYELNFPRSPRIRKRFLLRRILEMLFFTQLQVGLIQQWM
VPTIQNSMKPFKDMDYSRIIERLLKLAVPNHLIWLIFFYWLFHSCLNAVA
ELMQFGDREFYRDWWNSESVTYFWQNWNIPVHKWCIRHFYKPMLRRGSSK
WMARTGVFLASAFFHEYLVSVPLRMFRLWAFTGMMAQIPLAWFVGRFFQG
NYGNAAVWLSLIIGQPIAVLMYVHDYYVLNYEAPAAEA*

SEQ ID NO: 4

MELSDANLQTLTEYLKKTLDPDPAIRRPAEKFLESVEGNQNYPLLLLTLL
EKSQDNVIKVCASVTFKNYIKRNWRIVEDEPNKICEADRVAIKANIVHLM
LSSPEQIQKQLSDAISIIGREDFPQKWPDLLTEMVNRFQSGDFHVINGVL
RTAHSLFKRYRHEFKSNELWTEIKLVLDAFALPLTNLFKATIELCSTHAN
DASALRILFSSLILISKLFYSLNFQDLPEFFEDNMETWMNNFHTLLTLDN
KLLQTDDEEEAGLLELLKSQICDNAALYAQKYDEEFQRYLPRFVTAIWNL
LVTTGQEVKYDLLVSNAIQFLASVCERPHYKNLFEDQNTLTSICEKVIVP
NMEFRAADEEAFEDNSEEYIRRDLEGSDIDTRRRAACDLVRGLCKFFEGP
VTGIFSGYVNSMLQEYAKNPSVNWKHKDAAIYLVTSLASKAQTQKHGITQ
ANELVNLTEFFVNHILPDLKSANVNEFPVLKADGIKYIMIFRNQVPKEHL
LVSIPLLINHLQAESIVVHTYAAHALERLFTMRGPNNATLFTAAEIAPFV
EILLTNLFKALTLPGSSENEYIMKAIMRSFSLLQEAIIPYIPTLITQLTQ
KLLAVSKNPSKPHFNHYMFEAICLSIRITCKANPAAVVNFEEALFLVFTE
ILQNDVQEFIPYVFQVMSLLLETHKNDIPSSYMALFPHLLQPVLWERTGN
IPALVRLLQAFLERGSNTIASAAADKIPGLLGVFQKLIASKANDHQGFYL
LNSIIEHMPPESVDQYRKQIFILLFQRLQNSKTTKFIKSFLVFINLYCIK
YGALALQEIFDGIQPKMFGMVLEKIIIPEIQKVSGNVEKKICAVGITKLL
TECPPMMDTEYTKLWTPLLQSLIGLFELPEDDTIPDEEHFIDIEDTPGYQ
TAFSQLAFAGKKEHDPVGQMVNNPKIHLAQSLNKLSTACPGRVPSMVSTS
LNAEALQYLQGYLQAASVTLL*

SEQ ID NO: 5

MISASRAAAARLVGAAASRGPTAARHQDSWNGLSHEAFRLVSRRDYASEA
IKGAVVGIDLGTTNSCVAVMEGKQAKVLENAEGARTTPSVVAFTADGERL
VGMPAKRQAVTNPNNTFYATKRLIGRRYDDPEVQKDIKNVPFKIVRASNG
DAWVEAHGKLYSPSQIGAFVLMKMKETAENYLGHTAKNAVITVPAYFNDS
QRQATKDAGQISGLNVLRVINEPTAAALAYGLDKSEDKVIAVYDLGGGTF
DISILEIQKGVFEVKSTNGDTFLGGEDFDQALLRHIVKEFKRETGVDLTK
DNMALQRVREAAEKAKCELSSSVQTDINLPYLTMDSSGPKHLNMKLTRAQ
FEGIVTDLIRRTIAPCQKAMQDAEVSKSDIGEVILVGGMTRMPKVQQTVQ
DLFGRAPSKAVNPDEAVAIGAAIQGGVLAGDVTDVLLLDVTPLSLGIETL
GGVFTKLINRNTTIPTKKSQVFSTAADGQTQVEIKVCQGEREMAGDNKLL
GQFTLIGIPPAPRGVPQIEVTFDIDANGIVHVSAKDKGTGREQQIVIQSS
GGLSKDDIENMVKNAEKYAEEDRRKKERVEAVNMAEGIIHDTETKMEEFK
DQLPADECNKLKEEISKMRELLARKDSETGENIRQAASSLQQASLKLFEM
AYKKMASEREGSGSSGTGEQKEDQKEEKQ*

SEQ ID NO: 6

MEKDGLCRADQQYECVAEIGEGAYGKVFKARDLKNGGRFVALKRVRVQTG
EEGMPLSTIREVAVLRHLETFEHPNVVRLFDVCTVSRTDRETKLTLVFEH
VDQDLTTYLDKVPEPGVPTETIKDMMFQLLRGLDFLHSRRVVHRDLKPQN
ILVTSSGQIKLADFGLARIYSFQMALTSVVVTLWYRAPEVLLQSSYATPV
DLWSVGCIFAEMFRRKPLFRGSSDVDQLGKILDVIGLPGEEDWPRDVALP
RQAFHSKSAQPIEKFVTDIDELGKDLLLKCLTFNPAKRISAYSALSHPYF
QDLERCKENLDSHLPPSQNTSELNTA*

SEQ ID NO: 7

METEQPEETFPNTETNGEFGKRPAEDMEEEQAFKRSRNTDEMVELRILLQ
SKNAGAVIGKGGKNIKALRTDYNASVSVPDSSGPERILSISADIETIGEI
LKKIIPTLEEGLQLPSPTATSQLPLESDAVECLNYQHYKGSDFDCELRLL
IHQSLAGGIIGVKGAKIKELRENTQTTIKLFQECCPHSTDRVVLIGGKPD
RVVECIKIILDLISESPIKGRAQPYDPNFYDETYDYGGFTMMFDDRRGRP
VGFPMRGRGGFDRMPPGRGGRPMPPSRRDYDDMSPRRGPPPPPPGRGGRG
GSRARNLPLPPPPPPRGGDLMAYDRRGRPGDRYDGMVGFSADETWDSAID
TWSPSEWQMAYEPQGGSGYDYSYAGGRGSYGDLGGPIITTQVTIPKDLAG
SIIGKGGQRIKQIRHESGASIKIDEPLEGSEDRIITITGTQDQIQNAQYL
LQNSVKQYADVEGF*

SEQ ID NO: 8

MAMSFEWPWQYRFPPFFTLQPNVDTRQKQLAAWCSLVLSFCRLHKQSSMT
VMEAQESPLFNNVKLQRKLPVESIQIVLEELRKKGNLEWLDKSKSSFLIM
WRRPEEWGKLIYQWVSRSGQNNSVFTLYELTNGEDTEDEEFHGLDEATLL
RALQALQQEHKAEIITVSDGRGVKFF*

SEQ ID NO: 9

MGKEKTHINIVVIGHVDSGKSTTTGHLIYKCGGIDKRTIEKFEKEAAEMG
KGSFKYAWVLDKLKAERERGITIDISLWKFETSKYYVTIIDAPGRRDFIK
NMITGTSQADCAVLIVAAGVGEFEAGISKNGQTREHALLAYTLGVKQLIV
GVNKMDSTEPPYSQKRYEEIVKEVSTYIKKIGYNPDTVAFVPISGWNGDN
MLEPSANMPWFKGWKVTRKDGNASGTTLLEALDCILPPTRPTDKPLRLPL
QDVYKIGGIGTVPVGRVETGVLKPGMVVTFAPVNVTTEVKSVEMHHEALS
EALPGDNVGFNVKNVSVKDVRRGNVAGDSKNDPPMEAAGFTAQVIILNHP
GQISAGYAPVLDCHTAHIACKFAELKEKIDRRSGKKLEDGPKFLKSGDAA
IVDMVPGKPMCVESFSDYPPLGRFAVRDMRQTVAVGVIKAVDKKAAGAGK
VTKSAQKAQKAK*

SEQ ID NO: 10

MLLRSAGKLNVGTKKEDGESTAPTPRPKVLRCKCHHHCPEDSVNNICSTD
GYCFTMIEEDDSGLPVVTSGCLGLEGSDFQCRDTPIPHQRRSIECCTERN
ECNKDLHPTLPPLKNRDFVDGPIHHRALLISVTVCSLLLVLIILFCYFRY
KRQETRPRYSIGLEQDETYIPPGESLRDLIEQSQSSGSGSGLPLLVQRTI
AKQIQMVKQIGKGRYGEVWMGKWRGEKVAVKVFFTTEEASWFRETEIYQT
VLMRHENILGFIAADIKGTGSWTQLYLITDYHENGSLYDYLKSTTLDAKS
MLKLAYSSVSGLCHLHTEIFSTQGKPAIAHRDLKSKNILVKKNGTCCIAD
LGLAVKFISDTNEVDIPPNTRVGTKRYMPPEVLDESLNRNHFQSYIMADM
YSFGLILWEVARRCVSGGIVEEYQLPYHDLVPSDPSYEDMREIVCIKKLR
PSFPNRWSSDECLRQMGKLMTECWAHNPASRLTALRVKKTLAKMSESQDI
KL*

SEQ ID NO: 11

MLGTGPAAATTAATTSSNVSVLQQFASGLKSRNEETRAKAAKELQHYVTM
ELREMSQEESTRFYDQLNHHIFELVSSSDANERKGGILAIASLIGVEGGN
ATRIGRFANYLRNLLPSNDPVVMEMASKAIGRLAMAGDTFTAEYVEFEVK
RALEWLGADRNEGRRHAAVLVLRELAISVPTFFFQQVQPFFDNIFVAVWD
PKQAIREGAVAALRACLILTTQREPKEMQKPQWYRHTFEEAEKGFDETLA
KEKGMNRDDRIHGALLILNELVRISSMEGERLREEMEEITQQQLVHDKYC
KDLMGFGTKPRHITPFTSFQAVQPQQSNALVGLLGYSSHQGLMGFGTSPS
PAKSTLVESRCCRDLMEEKFDQVCQWVLKCRNSKNSLIQMTILNLLPRLA
AFRPSAFTDTQYLQDTMNHVLSCVKKEKERTAAFQALGLLSVAVRSEFKV
YLPRVLDIIRAALPPKDFAHKRQKAMQVDATVFTCISMLARAMGPGIQQD
IKELLEPMLAVGLSPALTAVLYDLSRQIPQLKKDIQDGLLKMLSLVLMHK
PLRHPGMPKGLAHQLASPGLTTLPEASDVGSITLALRTLGSFEFEGHSLT
QFVRRCADHFLNSEHKEIRMEAARTCSRLLTPSIHLISGHAHVVSQTAVQ
VVADVLSKLLVVGITDPDPDIRYCVLASLDERFDAHLAQAENLQALFVAL
NDQVFEIRELAICTVGRLSSMNPAFVMPFLRKMLIQILTELEHSGIGRIK
EQSARMLGHLVSNAPRLIRPYMEPILKALILKLKDPDPDPNPGVINNVLA
TIGELAQVSGLEMRKWVDELFIIIMDMLQDSSLLAKRQVALWTLGQLVAS
TGYVVEPYRKYPTLLEVLLNFLKTEQNQGTRREAIRVLGLLGALDPYKHK
VNIGMIDQSRDASAVSLSESKSSQDSSDYSTSEMLVNMGNLPLDEFYPAV
SMVALMRIFRDQSLSHHHTMVVQAITFIFKSLGLKCVQFLPQVMPTFLNV
IRVCDGAIREFLFQQLGMLVSFVKSHIRPYMDEIVTLMREFWVMNTSIQS
TIILLIEQIVVALGGEFKLYLPQLIPHMLRVFNEDNSPGRIVSIKLLAAI
QLFGANLDDYLHLLLPPIVKLFDAPEAPLPSRKAALETVDRLTESLDFTD
YASRIIHPLVRTLDQSPELRSTAMDTLSSLVFQLGKKYQIFIPMVNKVLV
RHRINHQRYDVLICRIVKGYTLADEEEDPLIYQHRMLRSGQGDALASGPV
ETGPMKKLHVSTINLQKAWGAARRVSKDDWLEWLRRLSLELLKDSSSPSL
RSCWALAQAYNPMARDLFNAAFVSCWSELNEDQQDELIRSIELALTSQDI
AEVTQTLLNLAEFMEHSDKGPLPLRDDNGIVLLGERAAKCRAYAKALHYK
ELEFQKGPTPAILESLISINNKLQQPEAAAGVLEYAMKHFGELEIQATWY
EKLHEWEDALVAYDKKMDTNKDDPELMLGRMRCLEALGEWGQLHQQCCEK
WTLVNDETQAKMARMAAAAAWGLGQWDSMEEYTCMIPRDTHDGAFYRAVL
ALHQDLFSLAQQCIDKARDLLDAELTAMAGESYSRAYGAMVSCHMLSELE
EVIQYKLVPERREIIRQIWWERLQGCQRIVEDWQKILMVRSLVVSPHEDM
RTWLKYASLCGKSGRLALAHKTLVLLLGVDPSRQLDHPLPTVHPQVTYAY
MKNMWKSARKIDAFQHMQHFVQTMQQQAQHAIATEDQQHKQELHKLMARC
FLKLGEWQLNLQGINESTIPKVLQYYSAATEHDRSWYKAWHAWAVMNFEA
VLHYKHQNQARDEKKKLRHASGANITNATTAATTAATATTTASTEGSNSE
SEAESTENSPTPSPLQKKVTEDLSKTLLMYTVPAVQGFFRSISLSRGNNL
QDTLRVLTLWFDYGHWPDVNEALVEGVKAIQIDTWLQVIPQLIARIDTPR
PLVGRLIHQLLTDTGRYHPQALIYPLTVASKSTTTARHNAANKILKNMCE
HSNTLVQQAMMVSEELIRVAILWHEMWHEGLEEASRLYFGERNVKGMFEV
LEPLHAMMERGPQTLKETSFNQAYGRDLMEAQEWCRKYMKSGNVKDLTQA
WDLYYHVFRRISKQLPQLTSLELQYVSPKLLMCRDLELAVPGTYDPNQPI
IRIQSIAPSLQVITSKQRPRKLTLMGSNGHEFVFLLKGHEDLRQDERVMQ
LFGLVNTLLANDPTSLRKNLSIQRYAVIPLSTNSGLIGWVPHCDTLHALI
RDYREKKKILLNIEHRIMLRMAPDYDHLTLMQKVEVFEHAVNNTAGDDLA
KLLWLKSPSSEVWFDRRTNYTRSLAVMSMVGYILGLGDRHPSNLMLDRLS
GKILHIDFGDCFEVAMTREKFPEKIPFRLTRMLTNAMEVTGLDGNYRITC
HTVMEVLREHKDSVMAVLEAFVYDPLLNWRLMDTNTKGNKRSRTRTDSYS
AGQSVEILDGVELGEPAHKKTGTTVPESIHSFIGDGLVKPEALNKKAIQI
TNRVRDKLTGRDFSHDDTLDVPTQVELLIKQATSHENLCQCYIGWCPFW*

SEQ ID NO: 12

MPGEATETVPATEQELPQPQAETGSGTESDSDESVPELEEQDSTQATTQQ
AQLAAAAEIDEEPVSKAKQSRSEKKARKAMSKLGLRQVTGVTRVTIRKSK
NILFVITKPDVYKSPASDTYIVFGEAKIEDLSQQAQLAAAEKFKVQGEAV
SNIQENTQTPTVQEESEEEEVDETGVEVKDIELVMSQANVSRAKAVRALK
NNSNDIVNAIMELTM*

SEQ ID NO: 13

MEVPQPEPAPGSALSPAGVCGGAQRPGHLPGLLLGSHGLLGSPVRAAASS
PVTTLTQTMHDLAGLGSETPKSQVGTLLFRSRSRLTHLSLSRRASESSLS
SESSESSDAGLCMDSPSPMDPHMAEQTFEQAIQAASRIIRNEQFAIRRFQ
SMPDGFVFKMPWKPTHPSSTHALAEWASRREAFAQRPSSAPDLMCLSPDR
KMEVEELSPLALGRFSLTPAEGDTEEDDGFVDILESDLKDDDAVPPGMES
LISAPLVKTLEKEEEKDLVMYSKCQRLFRSPSMPCSVIRPILKRLERPQD
RDTPVQNKRRRSVTPPEEQQEAEEPKARVLRSKSLCHDEIENLLDSDHRE
LIGDYSKAFLLQTVDGKHQDLKYISPETMVALLTGKFSNIVDKFVIVDCR
YPYEYEGGHIKTAVNLPLERDAESFLLKSPIAPCSLDKRVILEFHCEFSS
ERGPRMCRFIRERDRAVNDYPSLYYPEMYILKGGYKEFFPQHPNFCEPQD
YRPMNHEAFKDELKTFRLKTRSWAGERSRRELCSRLQDQ*

SEQ ID NO: 14

MGDLKDDKDSPKKNKGKERRDLDDLKKEVAMTEHKMSVEEVCRKYNTDCV
QGLTHSKAQEILARDGPNALTPPPTTPEWVKFCRQLFGGFSILLWIGAIL
CFLAYGIQAGTEDDPSGDNLYLGIVLAAVVIITGCFSYYQEAKSSKIMES
FKNMVPQQALVIREGEKMQVNAEEVVVGDLVEIKGGDRVPADLRIISAHG
CKVDNSSLTGESEPQTRSPDCTHDNPLETRNITFFSTNCVEGTARGVVVA
TGDRTVMGRIATLASGLEVGKTPIAIEIEHFIQLITGVAVFLGVSFFILS
LILGYTWLEAVIFLIGIIVANVPEGLLATVTVCLTLTAKRMARKNCLVKN
LEAVETLGSTSTICSDKTGTLTQNRMTVAHMWFDNQIHEADTTEDQSGTS
FDKSSHTWVALSHIAGLCNRAVFKGGQDNIPVLKRDVAGDASESALLKCI
ELSSGSVKLMRERNKKVAEIPFNSTNKYQLSIHETEDPNDNRYLLVMKGA
PERILDRCSTILLQGKEQPLDEEMKEAFQNAYLELGGLGERVLGECHYYL
PEEQFPKGFAFDCDDVNFTTDNLCFVGLMSMIDPPRAAVPDAVGKCRSAG
IKVIMVTGDHPITAKAIAKGVGIISEGNETVEDIAARLNIPVSQVNPRDA
KACVIHGTDLKDFTSEQIDEILQNHTEIVFARTSPQQKLIIVEGCQRQGA
IVAVTGSGVNDSPALKKADIGVAMGIAGSDVSKQAADMILLDDNFASIVT
GVEEGRLIFDNLKKSIAYTLTSNIPEITPFLLFIMANIPLPLGTITILCI
DLGTDMVPAISLAYEAAESDIMKRQPRNPRTDKLVNERLISMAYGQIGMI
QALGGFFSYFVILAENGFLPGNLVGIRLNWDDRTVNDLEDSYGQQWTYEQ
RKVVEFTCHTAFFVSIVVVQWADLIICKTRRNSVFQQGMKNKILIFGLFE
ETALAAFLSYCPGMDVALRMYPLKPSWWFCAFPYSFLIFVYDEIRKLILR
RNPGGWVEKETYY*

SEQ ID NO: 15

MPNSEPASLLELFNSIATQGELVRSLKAGNASKDEIDSAVKMLVSLKMSY
KAAAGEDYKADCPPGNPAPTSNHGPDATEAEEDFVDPWTVQTSSAKGIDY
DKLIVRFGSSKIDKELINRIERATGQRPHHFLRRGIFFSHRDMNQVLDAY
ENKKPFYLYTGRGPSSEAMHVGHLIPFIFTKWLQDVFNVPLVIQMTDDEK
YLWKDLTLDQAYSYAVENAKDIIACGFDINKTFIFSDLDYMGMSSGFYKN
VVKIQKHVTFNQVKGIFGFTDSDCIGKISFPAIQAAPSFSNSFPQIFRDR
TDIQCLIPCAIDQDPYFRMTRDVAPRIGYPKPALLHSTFFPALQGAQTKM
SASDPNSSIFLTDTAKQIKTKVNKHAFSGGRDTIEEHRQFGGNCDVDVSF
MYLTFFLEDDDKLEQIRKDYTSGAMLTGELKKALIEVLQPLIAEHIQARR
KEVTDEIVKEFMTPRKLSFDFQ*

SEQ ID NO: 16

MAKAKKVGARRKASGAPAGARGGPAKANSNPFEVKVNRQKFQILGRKTRH
DVGLPGVSRARALRKRTQTLLKEYKERDKSNVFRDKRFGEYNSNMSPEEK
MMKRFALEQQRHHEKKSIYNLNEDEELTHYGQSLADIEKHNDIVDSDSDA
EDRGTLSAELTAAHFGGGGGLLHKKTQQEGEEREKPKSRKELIEELIAKS
KQEKRERQAQREDALELTEKLDQDWKEIQTLLSHKTPKSENRDKKEKPKP
DAYDMMVRELGFEMKAQPSNRMKTEAELAKEEQEHLRKLEAERLRRMLGK
DEDENVKKPKHMSADDLNDGFVLDKDDRRLLSYKDGKMNVEEDVQEEQSK
EASDPESNEEEGDSSGGEDTEESDSPDSHSDLESNVESEEENEKPAKEQR
QTPGKGLISGKERAGKATRDELPYTFAAPESYEELRSLLLGRSMEEQLLV
VERIQKCNHPSLAEGNKAKLEKLFGFLLEYVGDLATDDPPDLTVIDKLVV
HLYHLCQMFPESASDAIKFVLRDAMHEMEEMIETKGRAALPGLDVLIYLK
ITGLLFPTSDFWHPVVTPALVCLSQLLTKCPILSLQDVVKGLFVCCLFLE
YVALSQRFIPELINFLLGILYIATPNKASQGSTLVHPFRALGKNSELLVV
SAREDVATWQQSSLSLRWASRLRAPTSTEANHIRLSCLAVGLALLKRCVL
MYGSLPSFHAIMGPLQALLTDHLADCSHPQELQELCQSTLTEMESQKQLC
RPLTCEKSKPVPLKLFTPRLVKVLEFGRKQGSSKEEQERKRLIHKHKREF
KGAVREIRKDNQFLARMQLSEIMERDAERKRKVKQLFNSLATQEGEWKAL
KRKKFKK*

SEQ ID NO: 17

MNPFWSMSTSSVRKRSEGEEKTLTGDVKTSPPRTAPKKQLPSIPKNALPI
TKPTSPAPAAQSTNGTHASYGPFYLEYSLLAEFTLVVKQKLPGVYVQPSY
RSALMWFGVIFIRHGLYQDGVFKFTVYIPDNYPDGDCPRLVFDIPVFHPL
VDPTSGELDVKRAFAKWRRNHNHIWQVLMYARRVFYKIDTASPLNPEAAV
LYEKDIQLFRSKVVDSVKVCTARLFDQPKIEDPYAISFSPWNPSVHDEAR
EKMLTQKKKPEEQHNEKSVHVAGLSWVKPGSVQPFSKEEKTVAT*

SEQ ID NO: 18

MAEEGIAAGGVMDVNTALQEVLKTALIHDGLARGIREAAKALDKRQAHLC
VLASNCDEPMYVEKLVEALCAEHQINLIKVDDNKKLGEWVGLCKIDREGK
PRKVVGCSCVVVKDYGKESQAKDVIEEYFKCKK*

SEQ ID NO: 19

MADFGISAGQFVAVVWDKSSPVEALKGLVDKLQALTGNEGRVSVENIKQL
LQSAHKESSFDIILSGLVPGSTTLHSAEILAEIARILRPGGCLFLKEPVE
TAVDNNSKVKTASKLCSALTLSGLVEVKELQREPLTPEEVQSVREHLGHE
SDNLLFVQITGKKPNFEVGSSRQLKLSITKKSSPSVKPAVDPAAAKLWTL
SANDMEDDSMDLIDSDELLDPEDLKKPDPASLRAASCGEGKKRKACKNCT
CGLAEELEKEKSREQMSSQPKSACGNCYLGDAFRCASCPYLGMPAFKPGE
KVLLSDSNLHDA*

SEQ ID NO: 20

MADAFGDELFSVFEGDSTTAAGTKKDKEKDKGKWKGPPGSADKAGKRFDG
KLQSESTNNGKNKRDVDFEGTDEPIFGKKPRIEESITEDLSLADLMPRVK
VQSVETVEGCTHEVALPAEEDYLPLKPRVGKAAKEYPFILDAFQREAIQC
VDNNQSVLVSAHTSAGKTVCAEYAIALALREKQRVIFTSPIKALSNQKYR
EMYEEFQDVGLMTGDVTINPTASCLVMTTEILRSMLYRGSEVMREVAWVI
FDEIHYMRDSERGVVWEETIILLPDNVHYVFLSATIPNARQFAEWICHLH
KQPCHVIYTDYRPTPLQHYIFPAGGDGLHLVVDENGDFREDNFNTAMQVL
RDAGDLAKGDQKGRKGGTKGPSNVFKIVKMIMERNFQPVIIFSFSKKDCE
AYALQMTKLDFNTDEEKKMVEEVFSNAIDCLSDEDKKLPQVEHVLPLLKR
GIGIHHGGLLPILKETIEILFSEGLIKALFATETFAMGINMPARTVLFTN
ARKFDGKDFRWISSGEYIQMSGRAGRRGMDDRGIVILMVDEKMSPTIGKQ
LLKGSADPLNSAFHLTYNMVLNLLRVEEINPEYMLEKSFYQFQHYRAIPG
VVEKVKNSEEQYNKIVIPNEESVVIYYKIRQQLAKLGKEIEEYIHKPKYC
LPFLQPGRLVKVKNEGDDFGWGVVVNFSKKSNVKPNSGELDPLYVVEVLL
RCSKESLKNSATEAAKPAKPDEKGEMQVVPVLVHLLSAISSVRLYIPKDL
RPVDNRQSVLKSIQEVQKRFPDGIPLLDPIDDMGIQDQGLKKVIQRVEAF
EHRMYSHPLHNDPNLETVYTLCEKKAQIAIDIKSAKRELKKARTVLQMDE
LKCRKRVLRRLGFATSSDVIEMKGRVACEISSADELLLTEMMFNGLFNDL
SAEQATALLSCFVFQENSSEMPKLTEQLAGPLRQMQECAKRIAKVSAEAK
LEIDEETYLSSFKPHLMDVVYTWATGATFAHICKMTDVFEGSIIRCMRRL
EELLRQMCQAAKAIGNTELENKFAEGITKIKRDIVFAASLYL*

SEQ ID NO: 21

MAAAGARPVELGFAESAPAWRLRSEQFPSKVGGRPAWLGAAGLPGPQALA
CELCGRPLSFLLQVYAPLPGRPDAFHRCIFLFCCREQPCCAGLRVFRNQL
PRKNDFYSYEPPSENPPPETGESVCLQLKSGAHLCRVCGCLGPKTCSRCH
KAYYCSKEHQTLDWRLGHKQACAQPDHLDHIIPDHNFLFPEFEIVIETED
EIMPEVVEKEDYSEIIGSMGEALEEELDSMAKHESREDKIFQKFRTQIAL
EPEQILRYGRGIAPIWISGENIPQEKDIPDCPCGAKRILEFQVMPQLLNY
LKADRLGKSIDWGILAVFTCAESCSLGTGYTEEFVWKQDVTDTP*

SEQ ID NO: 22

MALIMEPVSKWSPSQVVDWMKGLDDCLQQYIKNFEREKISGDQLLRITHQ
ELEDLGVSRIGHQELILEAVDLLCALNYGLETENLKTLSHKLNASAKNLQ
NFITGRRRSGHYDGRTSRKLPNDFLTSVVDLIGAAKSLLAWLDRSPFAAV
TDYSVTRNNVIQLCLELTTIVQQDCTVYETENKILHVCKTLSGVCDHIIS
LSSDPLVSQSAHLEVIQLANIKPSEGLGMYIKSTYDGLHVITGTTENSPA
DRCKKIHAGDEVIQVNHQTVVGWQLKNLVNALREDPSGVILTLKKRPQSM
LTSAPALLKNMRWKPLALQPLIPRSPTSSVATPSSTISTPTKRDSSALQD
LYIPPPPAEPYIPRDEKGNLPCEDLRGHMVGKPVHKGSESPNSFLDQEYR
KRFNIVEEDTVLYCYEYEKGRSSSQGRRESTPTYGKLRPISMPVEYNWVG
DYEDPNKMKRDSRRENSLLRYMSNEKIAQEEYMFQRNSKKDTGKKSKNKG
DKSNSPTHYSLLPSLQMDALRQDIMGTPVPETTLYHTFQQSSLQHKSKKK
NKGPIAGKSKRRISCKDLGRGDCEGWLWKKKDAKSYFSQIKWKKYWFVLK
DASLYWYINEEDEKAEGFISLPEFKIDRASECRKKYAFKACHPKIKSFYF
AAEHLDDMNRWLNRINMLTAGYAERERIKQEQDYWSESDKEEADTPSTPK
QDSPPPPYDTYPRPPSMSCASPYVEAKHSRLSSTETSQSQSSHEEFRQEV
TGSSAVSPIRKTASQRRSWQDLIETPLTSSGLHYLQTLPLEDSVFSDSAA
ISPEHRRQSTLPTQKCHLQDHYGPYPLAESERMQVLNGNGGKPRSFTLPR
DSGFNHCCLNAPVSACDPQDDVQPPEVEEEEEEEEEEGEAAGENIGEKSE
SREKKLGDSLQDLYRALEQASLSPLGEHRISTKMEYKLSFIKRCNDPVMN
EKLHRLRILKSTLKAREGEVAIIDKVLDNPDLTSKEFQQWKQMYLDLFLD
ICQNTTSNDPLSISSEVDVITSSLAHTNSYIETHV*

SEQ ID NO: 23

MFDKTRLPYVALDVLCVLLASMPMAVLKLGQIYPFQRGFFCKDNSINYPY
HDSTVTSTVLILVGVGLPISSIILGETLSVYCNLLHSNSFIRNNYIATIY
KAIGTFLFGAAASQSLTDIAKYSIGRLRPHFLDVCDPDWSKINCSDGYIE
YYICRGNAERVKEGRLSFYSGHSSFSMYCMLFVALYLQARMKGDWARLLR
PTLQFGLVAVSIYVGLSRVSDYKHHWSDVLTGLIQGALVAILVAVYVSDF
FKERTSFKERKEEDSHTTLHETPTTGNHYPSNHQP*

SEQ ID NO: 24

MAASERRAFAHKINRTVAAEVRKQVSRERSGSPHSSRRCSSSLGVPLTEV
VEPLDFEDVLLSRPPDAEPGPLRDLVEFPADDLELLLQPRECRTTEPGIP
KDEKLDAQVRAAVEMYIEDWVIVHRRYQYLSAAYSPVTTDTQRERQKGLP
RQVFEQDASGDERSGPEDSVRKPLAGVTEGNEYEDTLTRNDSRRGSGSPE
DTPRSSGASSIFDLRNLAADSLLPSLLERAAPEDVDRRNETLRRQHRPPA
LLTLYPAPDEDEAVERCSRPEPPREHFGQRILVKCLSLKFEIEIEPIFGI
LALYDVREKKKISENFYFDLNSDSMKGLLRAHGTHPAISTLARSAIFSVT
YPSPDIFLVIKLEKVLQQGDISECCEPYMVLKEVDTAKNKEKLEKLRLAA
EQFCTRLGRYRMPFAWTAVHLANIVSSAGQLDRDSDSEGERRPAWTDRRR
RGPQDRASSGDDACSFSGFRPATLTVTNFFKQEAERLSDEDLFKFLADMR
RPSSLLRRLRPVTAQLKIDISPAPENPHFCLSPELLHIKPYPDPRGRPTK
EILEFPAREVYAPRTSYRNLLYVYPHSLNFSSRQGSVRNLAVRVQYMTGE
DPSQALPVIFGKSSCSEFTREAFTPVVYHNKSPEFYEEFKLHLPACVTEN
HHLLFTFYHVSCQPRPGTALETPVGFTVALPGMRWVDGHKGVFSVELTAV
SSVHPQDPYLDKFFTLVHVLEEGAFPFRLKDTVLSEGNVEQELPASLAAL
RLASPEPLVAFSHHVLDKLVRLVIRPPIISGQIVNLGRGAFEAMAHVVSL
VHRSLEAAQDARGHCPQLAAYVHYAFRLPGTEPSLPDGAPPVTVQAATLA
RGSGRPASLYLARSKSISSSNPDLAVAPGSVDDEVSRILASKLLHEELAL
QWVVSSSAVREAILQHAWFFFQLMSMALHLLLGQRLDTPRKLRFPGRFLD
DITALVGSVGLEVITRVHKDVELAEHLNASLAFFLSDLLSLVDRGFVFSL
VRAHYKQVATRLQSSPNPAALLTLRMEFTRILCSHEHYVTLNLPCCPLSP
PASPSPSVSSTTSQSSTFSSQAPDPKVTSMFELSGPFRQQHFLAGLLLTE
LALALEPEAEGAFLLHKKAISAVHSLLCGHDTDPRYAEATVKARVAELYL
PLLSIARDTLPRLHDFAEGPGQRSRLASMLDSDTEGEGDIAGTINPSVAM
AIAGGPLAPGSRASISQGPPTASRAGCALSAESSRTLLACVLWVLKNTEP
ALLQRWATDLTLPQLGRLLDLLYLCLAAFEYKGKKAFERINSLTFKKSLD
MKARLEEAILGTIGARQEMVRRSRERSPFGNPENVRWRKSVTHWKQTSDR
VDKTKDENEMEALVEGNLATEASLVVLDTLEIIVQTVMLSEARESVLGAV
LKVVLYSLGSAQSALFLQHGLATQRALVSKFPELLFEEDTELCADLCLRL
LRHCGSRISTIRTHASASLYLLMRQNFEIGHNFARVKMQVTMSLSSLVGT
TQNFSEEHLRRSLKTILTYAEEDMGLRDSTFAEQVQDLMFNLHMILTDTV
KMKEHQEDPEMLIDLMYRIARGYQGSPDLRLTWLQNMAGKHAELGNHAEA
AQCMVHAAALVAEYLALLEDHRHLPVGCVSFQNISSNVLEESAISDDILS
PDEEGFCSGKHFTELGLVGLLEQAAGYFTMGGLYEAVNEVYKNLIPILEA
HRDYKKLAAVHGKLQEAFTKIMHQPQRVFGTYFRVGFYGAHFGDLDEQEF
VYKEPSITKLAEISHRLEACPCGWGWNGAWGCLGAGEFYTERFGDDVVEI
IKDSNPVDKSKLDSQKAYIQITYVEPYFDTYELKDRVTYFDRNYGLRTFL
FCTPFTPDGRAHGELPEQHKRKTLLSTDHAFPYIKTRIRVCHREETVLTP
VEVAIEDMQKKTRELAFATEQDPPDAKMLQMVLQGSVGPTVNQARLVEGG
CKPHGGLQGPLEVAQVFLAEIPEDPKLFRHHNKLRLCFKDFCKNPVCESL
SISHPLALRCEDALRKNKALIGPDQKEYHRELERNYCRLREALQPLLTQR
LPQLMAPTPPGLRNSLNRASFRKADL*

SEQ ID NO: 25

MSHHGGAPKASTWVVASRRSSTVSRAPERRPAEELNRTGPEGYSVGRGGR
WRGTSRPPEAVAAGHEELPLCFALKSHFVGAVIGRGGSKIKNIQSTTNTT
IQIIQEQPESLVKIFGSKAMQTKAKAVIDNFVKKLEENYNSECGIDTAFQ
PSVGKDGSTDNNVVAGDRPLIDWDQIREEGLRWQKTKWADLPPIKKNFYK
ESTATSAMSKVEADSWRKENFNITWDDLKDGEKRPIPNPTCTFDDAFQCY
PEVMENIKKAGFQKPTPIQSQAWPIVLQGIDLIGVAQTGTGKTLCYLMPG
FIHLVLQPSLKGQRNRPGMLVLTPTRELALQVEGECCKYSYKGLRSVCVY
GGGNRDEQIEELKKGVDIIIATPGRLNDLQMSNFVNLKNITYLVLDEADK
MLDMGFEPQIMKILLDVRPDRQTVMTSATWPHSVHRLAQSYLKEPMIVYV
GTLDLVAVSSVKQNIIVTTEEEKWSHMQTFLQSMSSTDKVIVFVSRKAVA
DHLSSDLILGNISVESLHGDREQRDREKALENFKTGKVRILIATDLASRG
LDVHDVTHVYNFDFPRNIEEYVHRIGRTGRAGRTGVSITTLTRNDWRVAS
ELINILERANQSIPEELVSMAERFEAHQRKREMERKMERPQGRPKKFH*

SEQ ID NO: 26

MVVTRSARAKASIQAASAESSGQKSFAANGIQAHPESSTGSDARTTAESQ
TTGKQSLIPRTPKARKRKSRTTGSLPKGTEPSTDGETSEAESNYSVSEHH
DTILRVTRRRQILIACSPVSSVRKKPKVTPTKESYTEEIVSEAESHVSGI
SRIVLPTEKTTGARRSKAKSLTDPSQESHTEAISDAETSSSDISFSGIAT
RRTRSMQRKLKAQTEKKDSKIVPGNEKQIVGTPVNSEDSDTRQTSHLQAR
SLSEINKPNFYNNDFDDDFSHRSSENILTVHEQANVESLKETKQNCKDLD
EDANGITDDGKEINEKSSQLKNLSELQDTSLQQLVSQRHSTPQNKNAVSV
HSNLNSEAVMKSLTQTFATVEVGRWNNNKKSPIKASDLTKFGDCGGSDDE
EESTVISVSEDMNSEGNVDFECDTKLYTSAPNTSQGKDNSVLLVLSSDES
QQSENSENEEDTLCFVENSGQRESLSGDTGSLSCDNALFVIDTTPGMSAD
KNFYLEEEDKASEVAIEEEKEEEEDEKSEEDSSDHDENEDEFSDEEDFLN
STKAKLLKLTSSSIDPGLSIKQLGGLYINFNADKLQSNKRTLTQIKEKKK
NELLQKAVITPDFEKNHCVPPYSESKYQLQKKRRKERQKTAGDGWFGMKA
PEMTNELKNDLKALKMRASMDPKRFYKKNDRDGFPKYFQIGTIVDNPADF
YHSRIPKKQRKRTIVEELLADSEFRRYNRRKYSEIMAEKAANAAGKKFRK
KKKFRN*

SEQ ID NO: 27

MPGPRRPAGSRLRLLLLLLLPPLLLLLRGSHAGNLTVAVVLPLANTSYPW
SWARVGPAVELALAQVKARPDLLPGWTVRTVLGSSENALGVCSDTAAPLA
AVDLKWEHNPAVFLGPGCVYAAAPVGRFTAHWRVPLLTAGAPALGFGVKD
EYALTTRAGPSYAKLGDFVAALHRRLGWERQALMLYAYRPGDEEHCFFLV
EGLFMRVRDRLNITVDHLEFAEDDLSHYTRLLRTMPRKGRVIYICSSPDA
FRTLMLLALEAGLCGEDYVFFHLDTFGQSLQGGQGPAPRRPWERGDGQDV
SARQAFQAAKIITYKDPDNPEYLEFLKQLKHLAYEQFNFTMEDGLVNTIP
ASFHDGLLLYIQAVTETLAHGGTVTDGENITQRMWNRSFQGVTGYLKIDS
SGDRETDFSLWDMDPENGAFRVVLNYNGTSQELVAVSGRKLNWPLGYPPP
DIPKCGFDNEDPACNQDHLSTLEVLALVGSLSLLGILIVSFFIYRKMQLE
KELASELWRVRWEDVEPSSLERHLRSAGSRLTLSGRGSNYGSLLTTEGQF
QVFAKTAYYKGNLVAVKRVNRKRIELTRKVLFELKHMRDVQNEHLTRFVG
ACTDPPNICILTEYCPRGSLQDILENESITLDWMFRYSLTNDIVKGMLFL
HNGAICSHGNLKSSNCVVDGRFVLKITDYGLESFRDLDPEQGHTVYAKKL
WTAPELLRMASPPVRGSQAGDVYSFGIILQEIALRSGVFHVEGLDLSPKE
IIERVTRGEQPPFRPSLALQSHLEELGLLMQRCWAEDPQERPPFQQIRLT
LRKFNRENSSNILDNLLSRMEQYANNLEELVEERTQAYLEEKRKAEALLY
QILPHSVAEQLKRGETVQAEAFDSVTIYFSDIVGFTALSAESTPMQVVTL
LNDLYTCFDAVIDNFDVKVETIGDAYMVVSGLPVRNGRLHACEVARMALA
LLDAVRSFRIRHRPQEQLRLRIGIHTGPVCAGVVGLKMPRYCLFGDTVNT
ASRMESNGEALKIHLSSETKAVLEEFGGFELELRGDVEMKGKGKVRTYWL
LGERGSSTRG*

SEQ ID NO: 28

GFLAARGGGGAATAVAAAAKMAEGLERVRISASELRGILATLAPQAGSRE
NMKELKEARPRKDNRRPDLEIYKPGLSRLRNKPKIKEPPGSEEFKDEIVN
DRDCSAVENGTQPVKDVCKELNNQEQNGPIDPENNRGQESFPRTAGQEDR
SLKIIKRTKKPDLQIYQPGRRLQTVSKESASRVEEEEVLNQVEQLRVEED
ECRGNVAKEEVANKPDRAEIEKSPGGGRVGAAKGEKGKRMGKGEGVRETH
DDPARGRPGSAKRYSRSDKRRNRYRTRSTSSAGSNNSAEGAGLTDNGCRR
RRQDRTKERPRLKKQVSVSSTDSLDEDRIDEPDGLGPRRSSERKRHLERN
WSGRGEGEQKNSAKEYRGTLRVTFDAEAMNKESPMVRSARDDMDRGKPDK
GLSSGGKGSEKQESKNPKQELRGRGRGILILPAHTTLSVNSAGSPESAPL
GPRLLFGSGSKGSRSWGRGGTTRRLWDPNNPDQKPALKTQTPQLHFLDTD
DEVSPTSWGDSRQAQASYYKFQNSDNPYYYPRTPGPASQYPYTGYNPLQY
PVGPTNGVYPGPYYPGYPTPSGQYVCSPLPTSTMSPEEVEQHMRNLQQQE
LHRLLRVADNQELQLSNLLSRDRISPEGLEKMAQLRAELLQLYERCILLD
IEFSDNQNVDQILWKNAFYQVIEKFRQLVKDPNVENPEQIRNRLLELLDE
GSDFFDSLLQKLQVTYKFKLEDYMDGLAIRSKPLRKTVKYALISAQRCMI
CQGDIARYREQASDTANYGKARSWYLKAQHIAPKNGRPYNQLALLAVYTR
RKLDAVYYYMRSLAASNPILTAKESLMSLFEETKRKAEQMEKKQHEEFDL
SPDQWRKGKKSTFRHVGDDTTRLEIWIHPSHPRSSQGTESGKDSEQENGL
GSLSPSDLNKRFILSFLHAHGKLFTRIGMETFPAVAEKVLKEFQVLLQHS
PSPIGSTRMLQLMTINMFAVHNSQLKDCFSEECRSVIQEQAAALGLAMFS
LLVRRCTCLLKESAKAQLSSPEDQDDQDDIKVSSFVPDLKELLPSVKVWS
DWMLGYPDTWNPPPTSLDLPSHVAVDVWSTLADFCNILTAVNQSEVPLYK
DPDDDLTLLILEEDRLLSGFVPLLAAPQDPCYVEKTSDKVIAADCKRVTV
LKYFLEALCGQEEPLLAFKGGKYVSVAPVPDTMGKEMGSQEGTRLEDEEE
DVVIEDFEEDSEAEGSGGEDDIRELRAKKLALARKIAEQQRRQEKIQAVL
EDHSQMRQMELEIRPLFLVPDTNGFIDHLASLARLLESRKYILVVPLTVI
NELDGLAKGQETDHRAGGYARVVQEKARKSIEFLEQRFESRDSCLRALTS
RGNELESIAFRSEDITGQLGNNDDLILSCCLHYCKDKAKDFMPASKEEPI
RLLREVVLLTDDRNLRVKALTRNVPVRDIPAFLTWAQVG*

SEQ ID NO: 29

MLPAAARPLWGPCLGLRAAAFRLARRQVPCVCAVRHMRSSGHQRCEALAG
APLDNAPKEYPPKIQQLVQDIASLTLLEISDLNELLKKTLKIQDVGLVPM
GGVMSGAVPAAAAQEAVEEDIPIAKERTHFTVRLTEAKPVDKVKLIKEIK
NYIQGINLVQAKKLVESLPQEIKANVAKAEAEKIKAALEAVGGTVVLE*

SEQ ID NO: 30

MALCNGDSKLENAGGDLKDCHHHYEGAVVILDAGAQYGKVIDRRVRELFV
QSEIEFPLETPAFAIKEQGFRAIIISGGPNSVYAEDAPWFDPAIFTIGKP
VLGICYGMQMMNKVFGGTVHKKSVREDGVFNISVDNTCSLFRGLQKEEVV
LLTHGDSVDKVADGFKVVARSGNIVAGIANESKKLYGAQFHPEVGLTENG
KVILKNFLYDIAGCSGTFTVQNRELECIREIKERVGTSKVLVLLSGGVDS
TVCTALLNRALNQEQVIAVHIDNGFMRKRESQSVEEALKKLGIQVKVINA
AHSFYNGTTTLPISDEDRTPRKRISKTLNMTTSPEEKRKIIGDTFVKIAN
EVIGEMNLKPEEVFLAQGTLRPDLIESASLVASGKAELIKTHHNDTELIR
KLREEGKVIEPLKDFHKDEVRILGRELGLPEELVSRHPFPGPGLAIRVIC
AEEPYICKDFPETNNILKIVADFSASVKKPHTLLQRVRACTTEEDQEKLM
QITSLHSLNAFLLPIKTVGVQGDCRSYSYVCGISSKDEPDWESLIFLARL
IPRMCHNVNRVVYIFGPPVKEPPTDVTPTFLTTGVLSTLRQADFEAHNIL
RESGYAGKISQMPVILTPLHFDRDPLQKQPSCQRSVVIRTFITSDFMTGI
PATPGNEIPVEVVLKMVTEIKKIPGISRIMYDLTSKPPGTTEWE*

SEQ ID NO: 31

MAASQVLGEKINILSGETVKAGDRDPLGNDCPEQDRLPQRSWRQKCASYV
LALRPWSFSASLTPVALGSALAYRSHGVLDPRLLVGCAVAVLAVHGAGNL
VNTYYDFSKGIDHKKSDDRTLVDRILEPQDVVRFGVFLYTLGCVCAACLY
YLSPLKLEHLALIYFGGLSGSFLYTGGIGFKYVALGDLIILITFGPLAVM
FAYAIQVGSLAIFPLVYAIPLALSTEAILHSNNTRDMESDREAGIVTLAI
LIGPTFSYILYNTLLFLPYLVFSILATHCTISLALPLLTIPMAFSLERQF
RSQAFNKLPQRTAKLNLLLGLFYVFGIILAPAGSLPKI*

SEQ ID NO: 32

DAEFRHDSGYEVHHQKLVFFAEDVGSNKGAIIGLMVGGVVIA

SEQ ID NO:33

DAEFRHDSGYEVHHQKLVFFAEDVGSNKGAIIGLMVGGVVIATVIVITLV
MLKKKQYTSIHHGVVEVDAAVTPPEERHLSKMQQNGYENPTYKFFEQMQN

What is claimed is:

1. A method to treat, prevent or ameliorate a neurodegenerative condition comprising administering to a subject in need thereof an effective amount of a modulator of a protein selected from the group consisting of the proteins disclosed in SEQ ID NOS:1-31.

2. The method of claim 1, wherein said condition is Alzheimer's Disease.

3. The method of claim 1, wherein said modulator inhibits a biological activity of said protein in said subject.

4. The method of claim 3, wherein said modulator comprises one or more antibodies or fragments thereof that bind said protein, wherein said one or more antibodies or fragments thereof inhibit a biological activity of said protein in said subject.

5. The method of claim 1, wherein said modulator enhances a biological activity of said protein in said subject.

6. The method of claim 1, wherein said modulator inhibits expression of a gene encoding said protein in said subject.

7. The method of claim 6, wherein said modulator comprises one or more substances selected from the group consisting of an antisense oligonucleotide, a triple-helix DNA, a ribozyme, an RNA aptamer, a siRNA, a double-stranded RNA, and a single-stranded RNA.

8. The method of claim 1, wherein said modulator enhances expression of a gene encoding said protein in said subject.

9. A method to treat, prevent or ameliorate a neurodegenerative condition comprising administering to a subject in need thereof a pharmaceutical composition comprising an effective amount of a modulator of a protein selected from the group consisting of the proteins disclosed in SEQ ID NOS:1-31.

10. The method of claim 9, wherein said condition is Alzheimer's Disease.

11. The method of claim 9, wherein said modulator inhibits a biological activity of said protein in said subject.

12. The method of claim 11, wherein said modulator comprises one or more antibodies that bind said protein, or fragments thereof, wherein said one or more antibodies or fragments thereof inhibit a biological activity of said protein in said subject.

13. The method of claim 9, wherein said modulator enhances a biological activity of said protein in said subject.

14. The method of claim 9, wherein said modulator inhibits expression of a gene encoding said protein in said subject.

15. The method of claim 14, wherein said modulator comprises one or more substances selected from the group consisting of an antisense oligonucleotide, a triple-helix DNA, a ribozyme, an RNA aptamer, a siRNA, a double-stranded RNA, and a single-stranded RNA.

16. The method of claim 9, wherein said modulator enhances expression of a gene encoding said protein in said subject.

17. A method to identify modulators useful to treat, prevent or ameliorate neurodegenerative conditions comprising assaying for the ability of a candidate modulator to modulate a biological activity of a protein selected from the group consisting of the proteins disclosed in SEQ ID NOS:1-31.

18. The method of claim 17, wherein said method further comprises assaying for the ability of an identified modulator to reverse the pathological effects observed in vitro or in vivo models of said conditions.

19. The method of claim 17, wherein said method further comprises assaying for the ability of an identified modulator to reverse the pathological effects observed in clinical studies with subjects with said conditions.

20. The method according to claim 17, wherein said condition is Alzheimer's Disease.

21. A method to identify a modulator useful to treat, prevent or ameliorate a neurodegenerative condition comprising assaying for the ability of a candidate modulator to modulate gene expression of a protein selected from the group consisting of the proteins disclosed in SEQ ID NOS:1-31.

22. The method according to claim 21, wherein said method further comprises assaying for the ability of a candidate modulator to reverse a pathological effect characteristic of the condition observed in an in vitro model or in vivo model of said condition.

23. The method according to claim 21, wherein said method further comprises assaying for the ability of an identified inhibitory modulator to reverse a pathological effect observed in clinical studies with subjects having said condition.

24. The method according to claim 21, wherein said condition is Alzheimer's Disease.

25. A pharmaceutical composition comprising a modulator of a protein selected from the group consisting of the proteins disclosed in SEQ ID NOS: 1-31 in an amount effective to treat, prevent or ameliorate a neurodegenerative condition in a subject in need thereof.

26. The pharmaceutical composition according to claim 25, wherein said condition is Alzheimer's Disease.

27. The pharmaceutical composition according to claim 25, wherein said modulator inhibits a biological activity of said protein.

28. The pharmaceutical composition of claim 25, wherein said modulator comprises one or more antibodies that bind said protein, or fragments thereof.

29. The pharmaceutical composition according to claim 25, wherein said modulator enhances a biological activity of said protein.

30. The pharmaceutical composition according to claim 25, wherein said modulator inhibits gene expression of said protein.

31. The pharmaceutical composition of claim 30, wherein said modulator comprises one or more substances selected from the group consisting of an antisense oligonucleotide, a triple-helix DNA, a ribozyme, an RNA aptamer, a siRNA, a double-stranded RNA, and a single-stranded RNA.

32. The pharmaceutical composition according to claim 25, wherein said modulator enhances gene expression of said protein.

33. A method to diagnose a subject suffering from a neurodegenerative condition that may be a suitable candidate for treatment with a modulator of a protein selected from the group consisting of proteins disclosed in SEQ ID NOS:1-31, comprising assaying the mRNA level whose translation provides any one or more of said proteins in a biological sample from said subject wherein a subject with an altered mRNA level compared to a control is a suitable candidate for modulator treatment.

34. A method to diagnose a subject suffering from a neurodegenerative condition who may be suitable candidates for treatment with a modulator of a protein selected from the group consisting of proteins disclosed in SEQ ID NOS:1-31, comprising detecting the level of any one or more of said proteins in a biological sample from said subject wherein subjects with altered levels compared to a control is a suitable candidate for modulator treatment.

35. A method to treat, prevent or ameliorate a neurodegenerative condition comprising:
(a) assaying for the level of an mRNA encoding a protein selected from the group consisting of the proteins disclosed in SEQ ID NOS:1-31 in a biological sample from a subject; and
(b) administering to a subject with altered levels of mRNA of said protein compared to controls a modulator to said protein in an amount sufficient to treat, prevent or ameliorate the pathological effect of said condition.

36. The method of claim 35, wherein said condition is Alzheimer's Disease.

37. The method of claim 35, wherein said modulator enhances the gene expression of said protein.

38. The method of claim 35, wherein said modulator inhibits the gene expression of said protein.

39. A method to treat, prevent or ameliorate a neurodegenerative condition comprising:
(a) assaying for the level of a protein selected from the group consisting of the proteins disclosed in SEQ ID NOS:1-31 in a biological sample from a subject; and
(b) administering to a subject with altered levels of said protein compared to a control a modulator of said protein in an amount sufficient to treat, prevent or ameliorate the pathological effects of said condition.

40. The method of claim 39, wherein said condition is Alzheimer's Disease.

41. The method of claim 39, wherein said modulator enhances a biological activity of said protein.

42. The method of claim 39, wherein said modulator inhibits a biological activity of said protein.

43. A diagnostic kit for detecting mRNA levels of a protein selected from the group consisting of the proteins disclosed in SEQ ID NOS:1-31 in a biological sample, said kit comprising:
(a) a polynucleotide encoding a polypeptide set forth in SEQ ID NOS:1-31 or a fragment thereof;
(b) a nucleotide sequence complementary to that of (a);
(c) a polypeptide of SEQ ID NOS:1-31 of the present invention encoded by the polynucleotide of (a);
(d) an antibody to the polypeptide of (c);
(e) an RNAi sequence complementary to that of (a),
wherein components (a), (b), (c), (d) or (e) may comprise a substantial component.

44. A diagnostic kit for detecting levels of a protein selected from the group consisting of the proteins disclosed in SEQ ID NOS:1-31 in a biological sample, said kit comprising:
(a) a polynucleotide of a polypeptide set forth in SEQ ID NOS:1-31 or a fragment thereof;
(b) a nucleotide sequence complementary to that of (a);
(c) a polypeptide of SEQ ID NOS:11-31 of the present invention encoded by the polynucleotide of (a);
(d) an antibody to the polypeptide of (c);
(e) an RNAi sequence complementary to that of (a),
wherein components (a), (b), (c), (d) or (e) may comprise a substantial component.
